(19) United States
(12) Patent Application Publication
(10) Pub. No.: US 2026/0058009 A1
(43) Pub. Date: Feb. 26, 2026

(54) SECURE HEALTH MANAGEMENT SYSTEM

(71) Applicant: Dexcom, Inc., San Diego, CA (US)

(72) Inventors: Aniel Alvarez, Miami, FL (US); Reinier Bao, Sunny Isles, FL (US); Jorge R. Barreras, Miami, FL (US); Nathanael Richard Paul, Knoxville, TN (US)

(21) Appl. No.: 19/313,680

(22) Filed: Aug. 28, 2025

Related U.S. Application Data
(63) Continuation of application No. 17/308,754, filed on May 5, 2021.
(60) Provisional application No. 63/021,591, filed on May 7, 2020.

(51) Int. Cl. G16H 40/63; H04W 12/041; H04W 12/069; H04W 12/08

(52) U.S. Cl. CPC G16H 40/63; H04W 12/041; H04W 12/069; H04W 12/08

(57) ABSTRACT

Techniques and protocols for establishing secure communications between a display device, a sensor system, and a server system are disclosed. In certain embodiments, the techniques and protocols include secure diabetes device identification techniques and protocols, user-centric mutual authentication techniques and protocols, and device-centric mutual authentication techniques and protocols.

### CROSS-REFERENCE TO RELATED APPLICATIONS

This application is a continuation of U.S. patent application Ser. No. 17/308,754, filed May 5, 2021, which claims benefit of and priority to U.S. Provisional Application 63/021,591, filed May 7, 2020, which are hereby assigned to the assignee hereof and hereby expressly incorporated by reference herein in their entireties as if fully set forth below and for all applicable purposes.

### BACKGROUND

### Field

The present application relates generally to medical devices such as analyte sensors, and more particularly to systems, devices, and methods related to secure communications between medical devices and other communication devices in a diabetes management system.

### Description of the Related Technology

Diabetes is a metabolic condition relating to the production or use of insulin by the body. Insulin is a hormone that allows the body to use glucose for energy, or store glucose as fat.

Diabetes mellitus is a disorder in which the pancreas cannot create sufficient insulin (Type I or insulin dependent) and/or in which insulin is not effective (Type 2 or non-insulin dependent). In the diabetic state, the victim suffers from high blood sugar, which causes an array of physiological derangements (kidney failure, skin ulcers, or bleeding into the vitreous of the eye) associated with the deterioration of small blood vessels. A hypoglycemic reaction (low blood sugar) may be induced by an inadvertent overdose of insulin, or after a normal dose of insulin or glucose-lowering agent accompanied by extraordinary exercise or insufficient food intake.

Conventionally, a diabetic patient carries a self-monitoring blood glucose (SMBG) monitor, which may require uncomfortable finger pricking methods. Due to the lack of comfort and convenience, a diabetic will normally only measure his or her glucose level two to four times per day. Unfortunately, these time intervals are spread so far apart that the diabetic will likely be alerted to a hyperglycemic or hypoglycemic condition too late, sometimes incurring dangerous side effects as a result. In fact, it is unlikely that a diabetic will take a timely SMBG value, and further the diabetic will not know if his blood glucose value is going up (higher) or down (lower), due to limitations of conventional methods.

Consequently, a variety of non-invasive, transdermal (e.g., transcutaneous) and/or implantable sensors are being developed for continuously detecting and/or quantifying blood glucose values. Generally, in a diabetes management system, these sensors wirelessly transmit raw or minimally processed data for subsequent display and/or analysis at one or more remote devices, which can include a display device, a server, or any other types of communication devices. A remote device, such as a display device, may then utilize a trusted software application (e.g., approved and/or provided by the manufacturer of the sensor), which takes the raw or minimally processed data and provides the user with information about the user's blood glucose levels. Because diabetes management systems using such implantable sensors can provide more up-to-date information to users, they may reduce the risk of a user failing to regulate the user's blood glucose levels.

Using a wireless connection between a transcutaneous analyte sensor and one or more remote devices based on certain existing wireless communication protocols, however, may expose the sensor and/or the remote devices to safety, integrity, privacy, and availability issues (e.g., sensor and/or remote devices may become unavailable as a result of malicious attacks, etc.). As an example, an attacker may use a malicious device that impersonates the sensor to connect with and send inaccurate data (e.g., inaccurate blood glucose levels) to a user's display device to cause harm to the user. In another example, an attacker may use a malicious device to impersonate the user's display device, or the software application, and execute the software application on the user's display device to gain access to the user's sensor. In such an example, the attacker may receive the user's sensor data (e.g. blood glucose levels), thereby, violating the patient's privacy. Also, in such an example, the attacker may transmit data to the sensor that may cause malfunction of the sensor or sensor electronics. For example, a malicious or an impersonated display device may inaccurately calibrate the sensor, thereby causing the sensor to provide inaccurate blood glucose measurements. Further, in the same example, the attacker may disrupt a communication session that the user has already established between the user's sensor and the user's own display device that executes a trusted software application. In certain other examples, a user themselves may use an unauthenticated software application, that may be executed on the user's own display device, to connect with the user's sensor. In such an example, the unauthenticated software application may not include the necessary safety measures needed to ensure the user's data security and safety.

Moreover, guidelines from governing entities (e.g., Federal Drug Administration (FDA)) may require stringent security (e.g., cybersecurity) protocols for medical devices that may require authentication of each of the devices described above, as well as any software application executing thereon, to help eliminate some of the safety, integrity, privacy, and availability issues described above.

This background is provided to introduce a brief context for the summary and detailed description that follow. This background is not intended to be an aid in determining the scope of the claimed subject matter nor be viewed as limiting the claimed subject matter to implementations that solve any or all of the disadvantages or problems presented above.

### SUMMARY

Certain embodiments of the present disclosure provide a method of performing a diabetes device identification protocol between a sensor system for measuring blood glucose levels and a display device, comprising: executing, at the display device, the diabetes device identification protocol for identifying the sensor system. The executing comprises: obtaining, at the display device, a token ID of the sensor system. The executing further comprises computing a first sensor system identifier based on the token ID. The executing further comprises receiving a second sensor system identifier from the sensor system. The executing further comprises determining whether the first sensor system identifier and the second sensor system identifier are the same. The executing further comprises identifying that the sensor system is associated with a user of the display device based upon determining that the first sensor system identifier and the second system identifier are the same. The method further comprises: in response to the identifying, receiving, at the display device, analyte data indicative of blood glucose levels from the sensor system.

In the above method, computing the first sensor system identifier comprises: executing a hash function to hash the token ID into a hash value; and executing a truncating function to truncate the hash value, the hash value being the first sensor system identifier. In the above method, computing the first sensor system identifier comprises: executing a truncating function to truncate the token ID resulting in a truncated token ID; and executing a hash function to hash the truncated token ID into a hash value, the hash value being the first sensor system identifier. In the above method, the display device is configured to communicate with the sensor system only if signals received from the sensor system have a received signal strength above a threshold. In the above method, each of the sensor system and the display device is configured to reduce transmission power when executing the diabetes device identification protocol and further configured to increase the transmission power for transmission of analyte data.

Certain embodiments of the present disclosure provide a method of secure wireless communications between a sensor system for measuring blood glucose levels and a display device, comprising: executing, at the sensor system, a device-centric mutual authentication protocol with the display device to verify whether the display device is trusted by a diabetes trust management system. The executing comprises: receiving one or more credentials of the display device to verify whether the display device is trusted by the diabetes trust management system, wherein the one or more credentials of the display device comprise a credential token of the display device. The executing further comprises verifying the one or more credentials of the display device, wherein the verifying comprises authenticating the credential token of the display device. The executing further comprises transmitting one or more credentials of the sensor system to the display device for verification of the one or more credentials of the sensor system by the display device. The method further comprises: determining, at the sensor system, that the display device is trusted by the diabetes trust management system based on the executing, and, after the executing is successful, transmitting, at the sensor system, analyte data to the display device.

In the above method, the credential token includes a signature and information, and wherein authenticating the credential token of the display comprises: decrypting, at the sensor system, the signature in the credential token using a public key of the diabetes trust management system; and authenticating the credential token upon determining that the decrypted signature is the same as the information or a hash of the information.

In the above method, the one or more credentials of the display device comprise a message signed by the display device; the message includes an unencrypted first portion and a signature; the signature corresponds to an encrypted first portion; the encrypted first portion is encrypted using a private key of the display device; and verifying the one or more credentials of the display device comprises verifying an integrity of the display device using the message by: decrypting the signature using the public key of the display device; and determining that the decrypted signature is the same as the unencrypted first portion or a hash of the unencrypted first portion.

Certain embodiments of the present disclosure provide a method of secure wireless communications between a sensor system for measuring blood glucose levels and a display device, comprising executing, at the sensor system, a user-centric mutual authentication protocol with the display device to verify whether the display device is trusted by a user of the sensor system, wherein executing the user-centric mutual authentication protocol comprises executing a password authenticated key exchange (PAKE) algorithm with the display device using a shared key. The method further comprises, after the executing is successful, determining, at the sensor system, that the display device is trusted by the user of the sensor system. The method further comprises transmitting, at the sensor system, analyte data to the display device based on the determining.

In the above method, the shared key is a pairing code associated with the sensor system. In the above method, executing the PAKE algorithm comprises deriving an authorization key. The above method further comprises executing a key verification protocol with the display device to verify whether the display device is also in possession of the authorization key. In the above method, executing the key verification protocol with the display device comprises: receiving, at the sensor system, a first challenge value; hashing, at the sensor system, the first challenge value to generate a first hash value using a hashing algorithm and the authorization key; transmitting, to the display device, the first hash value and a second challenge value for the display device to verify whether the sensor system is in possession of the authorization key; hashing, at the sensor system, the second challenge value to generate a third hash value using the hashing algorithm and the authorization key; receiving, at the sensor system, a fourth hash value from the display device; verifying, at the sensor system, whether the third hash value and the fourth hash value are the same; and determining, at the sensor system, that the display device is trusted by the user of the sensor system based on determining that the display device is in possession of the authorization key upon verifying that the third hash value and the fourth hash value are the same.

In the above method, executing the key verification protocol with the display device comprises: computing an obfuscation key by hashing the authorization key and a random number; computing a third hash value by hashing the obfuscation key; computing a fourth hash value by hashing the third hash value; receiving a second hash value from the display device; verifying whether the second hash value and the fourth hash value are the same; determining that the display device is trusted by the user of the sensor system based on determining that the display device is in possession of the authorization key upon verifying that the second hash value and the fourth hash value are the same; and transmitting, to the display device, the third hash value for the display device to verify whether the sensor system is trusted by the user based on verifying whether a first hash value, based on the authorization key and the random number, and the third hash value are the same.

In the above method, executing the key verification protocol with the display device comprises: computing an obfuscation key by hashing the authorization key and a random number; computing a third hash value by hashing the obfuscation key; computing a fourth hash value by hashing the third hash value; receiving a second hash value and a signed second hash value from the display device; verifying whether the second hash value and the fourth hash value are the same; determining that the display device is trusted by the user of the sensor system based on determining that the display device is in possession of the authorization key upon verifying that the second hash value and the fourth hash value are the same; verifying integrity of the display device by: decrypting the signed second hash value using a public key of the display device; and determining that the display device is in possession of a private key of the display device based on determining that a decrypted signed second hash value is the same as the second hash value; and transmitting, to the display device, the third hash value and a signed third hash value for the display device to verify: whether the sensor system is trusted by the user based on verifying whether a first hash value, based on the authorization key and the random number, and the third hash value are the same; and integrity of the sensor system based on the signed third hash value.

Certain embodiments of the present disclosure provide a method of secure wireless communications between a sensor system for measuring blood glucose levels and a display device, comprising: executing, at the display device, a device-centric mutual authentication protocol with the sensor system to verify whether the sensor system is trusted by a diabetes trust management system. Executing the device-centric mutual authentication protocol with the sensor system comprises: transmitting, to the sensor system, one or more credentials of the display device, wherein the one or more credentials of the display device comprise a credential token of the display device. The executing further comprises receiving one or more credentials of the sensor system to verify whether the sensor system is trusted by the diabetes trust management system, wherein the one or more credentials of the sensor system comprise a credential token of the sensor system. The executing further comprises verifying the one or more credentials of the sensor system, wherein the verifying comprises authenticating the credential token of the sensor system. The method further comprises, after the executing is successful, determining, at the display device, that the sensor system is trusted by the diabetes trust management system. The method further comprises, receiving, at the display device, analyte data from the sensor system based on the determining.

In the above method, the credential token includes a signature and information, and wherein authenticating the credential token of the sensory system comprises: decrypting the signature in the credential token using a public key of the diabetes trust management system; authenticating the credential token upon determining that the decrypted signature is the same as the information or a hash of the information.

In the above method, the one or more credentials of the sensory system comprise a message signed by the sensory system; the message includes a unencrypted first portion and a signature; the signature corresponds to an encrypted first portion; then encrypted first portion is encrypted using a private key of the sensory system; verifying the one or more credentials of the sensory system comprises verifying an integrity of the sensory system using the message by: decrypting the signature using the public key of the sensory system; determining that the decrypted signature is the same as the unencrypted portion or a hash of the unencrypted portion.

The above method further comprises: engaging in a cryptographic key exchange algorithm with a server system prior to executing the device-centric mutual authentication protocol with the sensor system; and receiving the credential token of the display device from the server system, wherein the credential token of the display device is signed by the diabetes trust management system.

Certain embodiments of the present disclosure provide a method of secure wireless communications between a sensor system for measuring blood glucose levels and a display device, comprising: computing, at the sensor system, a sensor system identifier based on information associated with the sensor system. The method further comprises transmitting the sensor system identifier to the display device to identify whether the sensor system is associated with a user of the display device. The method further comprises upon identifying that the sensor system is associated with the user of the display device, executing, at the sensor system, a device-centric mutual authentication protocol with the display device to verify whether the display device is trusted by a diabetes trust management system. The method further comprises determining, at the sensor system, that the display device is trusted by the diabetes trust management system. The method further comprises executing, at the sensor system, a user-centric mutual authentication protocol with the display device to verify whether the display device is trusted by a user of the sensor system. The method further comprises determining, at the sensor system, that the display device is trusted by the user of the sensor system. The method further comprises transmitting analyte data to the display device over a secure connection, wherein the secure connection is secured using an encryption key.

In the above method, determining, at the sensor system, that the display device is trusted by the diabetes trust management system comprises: executing, at the sensor system, a device-centric mutual authentication protocol with the display device to verify whether the display device is trusted by the diabetes trust management system, wherein the executing comprises: receiving one or more credentials of the display device to verify whether the display device is trusted by the diabetes trust management system, wherein the one or more credentials of the display device comprise a credential token of the display device; verifying the one or more credentials of the display device, wherein the verifying comprises authenticating the credential token of the display device; and transmitting one or more credentials of the sensor system to the display device for verification of the one or more credentials of the sensor system by the display device; determining, at the sensor system, that the display device is trusted by the diabetes trust management system based on the executing.

In the above method, the credential token includes a signature and information, and wherein authenticating the credential token of the display comprises: decrypting, at the sensor system, the signature in the credential token using a public key of the diabetes trust management system; and authenticating the credential token upon determining that the decrypted signature is the same as the information or a hash of the information.

In the above method, the one or more credentials of the display device comprise a message signed by the display device; the message includes an unencrypted first portion and a signature; the signature corresponds to an encrypted first portion; the encrypted first portion is encrypted using a private key of the display device; and verifying the one or more credentials of the display device comprises verifying an integrity of the display device using the message by: decrypting the signature using the public key of the display device; and determining that the decrypted signature is the same as the unencrypted first portion or a hash of the unencrypted first portion.

In the above method, determining that the display device is trusted by the user of the sensor system comprises: executing, at the sensor system, the user-centric mutual authentication protocol with the display device to verify whether the display device is trusted by the user of the sensor system, wherein executing the user-centric mutual authentication protocol comprises executing a password authenticated key exchange (PAKE) algorithm with the display device using a shared key; and after the executing is successful, determining, at the sensor system, that the display device is trusted by the user of the sensor system. In the above method, the shared key is a pairing code associated with the sensor system. In the above method, executing the PAKE algorithm comprises deriving an authorization key. The above method further comprises executing a key verification protocol with the display device to verify whether the display device is also in possession of the authorization key.

In the above method, executing the key verification protocol with the display device comprises: receiving, at the sensor system, a first challenge value; hashing, at the sensor system, the first challenge value to generate a first hash value using a hashing algorithm and the authorization key; transmitting, to the display device, the first hash value and a second challenge value for the display device to verify whether the sensor system is in possession of the authorization key; hashing, at the sensor system, the second challenge value to generate a third hash value using the hashing algorithm and the authorization key; receiving, at the sensor system, a fourth hash value from the display device; verifying, at the sensor system, whether the third hash value and the fourth hash value are the same; and determining, at the sensor system, that the display device is trusted by the user of the sensor system based on determining that the display device is in possession of the authorization key upon verifying that the third hash value and the fourth hash value are the same.

In the above method, executing the key verification protocol with the display device comprises: computing an obfuscation key by hashing the authorization key and a random number; computing a third hash value by hashing the obfuscation key; computing a fourth hash value by hashing the third hash value; receiving a second hash value from the display device; verifying whether the second hash value and the fourth hash value are the same; determining that the display device is trusted by the user of the sensor system based on determining that the display device is in possession of the authorization key upon verifying that the second hash value and the fourth hash value are the same; and transmitting, to the display device, the third hash value for the display device to verify whether the sensor system is trusted by the user based on verifying whether a first hash value, based on the authorization key and the random number, and the third hash value are the same.

In the above method, executing the key verification protocol with the display device comprises: computing an obfuscation key by hashing the authorization key and a random number; computing a third hash value by hashing the obfuscation key; computing a fourth hash value by hashing the third hash value; receiving a second hash value and a signed second hash value from the display device; verifying whether the second hash value and the fourth hash value are the same; determining that the display device is trusted by the user of the sensor system based on determining that the display device is in possession of the authorization key upon verifying that the second hash value and the fourth hash value are the same; verifying integrity of the display device by: decrypting the signed second hash value using a public key of the display device; and determining that the display device is in possession of a private key of the display device based on determining that a decrypted signed second hash value is the same as the second hash value; and transmitting, to the display device, the third hash value and a signed third hash value for the display device to verify: whether the sensor system is trusted by the user based on verifying whether a first hash value, based on the authorization key and the random number, and the third hash value are the same; and integrity of the sensor system based on the signed third hash value.

Certain embodiments of the present disclosure provide a method of secure wireless communications between a sensor system for measuring blood glucose levels and a display device, comprising: computing, at the sensor system, a sensor system identifier based on information associated with the sensor system. The method further comprises transmitting the sensor system identifier to the display device to identify whether the sensor system is associated with a user of the display device. The method further comprises executing, at the sensor system, a mutual authentication protocol with the display device to verify whether the display device is trusted by a diabetes trust management system and a user of the sensor system. The method further comprises determining, at the sensor system, that the display device is trusted by the diabetes trust management system and the user of the sensor system, based on the executing. The method further comprises transmitting analyte data to the display device over a secure connection, wherein the secure connection is secured using an encryption key.

In the above method, executing the mutual authentication protocol with the display device comprises: executing a password authenticated key exchange (PAKE) algorithm with the display device using a shared key, wherein executing the PAKE algorithm further comprises: transmitting, at the sensor system, a signed credential token of the sensor system to the display device to allow the display device to verify whether the sensor system is trusted by the diabetes management system; and receiving, at the sensor system, a signed credential token of the display device to verify whether the display device is trusted by the diabetes management system. In the above method, determining that the display device is trusted by the diabetes trust management system and the user of the sensor system is based on a successful execution of the PAKE algorithm.

Certain embodiments of the present disclosure provide a method of secure wireless communications between a sensor system for measuring blood glucose levels and a display device, comprising executing, at the sensor system, a user-centric mutual authentication protocol with the display device to verify whether the display device is trusted by a user of the sensor system, wherein executing the user-centric mutual authentication protocol comprises executing a password authenticated key exchange (PAKE) algorithm with the display device using a shared key, and wherein executing the PAKE algorithm comprises deriving an authorization key. After determining that the display device is trusted by the user of the sensor system the method further comprises, executing, at the sensor system, a device-centric mutual authentication protocol with the display device to verify whether the display device is trusted by a diabetes trust management system, wherein the executing comprises: receiving one or more credentials from the display device to verify whether the display device is trusted by the diabetes trust management system, wherein the one or more credentials of the display device comprise a credential token of the display device. The executing further comprises verifying the one or more credentials of the display device, wherein the verifying comprises authenticating the credential token of the display device. The executing further comprises transmitting one or more credentials of the sensor system to the display device for verification of the one or more credentials of the sensor system by the display device. The executing further comprises determining, at the sensor system, that the display device is trusted by the diabetes trust management system based on the executing. The method further comprises after executing the device-centric mutual authentication protocol is successful, transmitting, at the sensor system, analyte data to the display device.

The above method further comprises: prior to executing a device-centric mutual authentication protocol executing, at the sensor system, a key verification protocol with the display device to verify whether the display device is also in possession of the authorization key, wherein executing the key verification protocol with the display device comprises: receiving, at the sensor system, a first challenge value; hashing, at the sensor system, the first challenge value to generate a first hash value using a hashing algorithm and the authorization key; transmitting, to the display device, the first hash value and a second challenge value for the display device to verify whether the sensor system is in possession of the authorization key; hashing, at the sensor system, the second challenge value to generate a third hash value using the hashing algorithm and the authorization key; receiving, at the sensor system, a fourth hash value from the display device; verifying, at the sensor system, whether the third hash value and the fourth hash value are the same; and determining, at the sensor system, that the display device is trusted by the user of the sensor system based on determining that the display device is in possession of the authorization key upon verifying that the third hash value and the fourth hash value are the same.

In the above method, the one or more credentials of the display device comprise an issuer credential token of an issuer of the credential token of the display device, and wherein verifying the one or more credentials of the display device comprises authenticating the issuer credential token. The above method further comprises: transmitting a first challenge data to the display device; receiving a signed version of the first challenge data including a digital signature generated by the display device using a private key associated with the display device; and verifying authenticity of the digital signature by using a public key associated with the display device to decrypt the digital signature. In the above method, transmitting the analyte data to the display device comprises: encrypting the analyte data with the authorization key or a portion of the authorization key.

In the above method, the sensor system receives communication encrypted with the authorization key from a second display device without having engaged in the user-centric authentication protocol with the second display device, and wherein the second display device is configured with the authorization key by the display device. In the above method, transmitting the analyte data to the display device comprises: generating a first message authentication code (MAC) using the analyte data, a MAC algorithm, and the authorization key or a portion of the portion of the authorization key; and transmitting the analyte data with the first MAC to the display device, wherein the display device: receives the analyte data and the first MAC; uses the analyte data and the authorization key or a portion of the authorization key to generate a second MAC; and verifies authenticity and integrity of the analyte data by determining that the first MAC and the second MAC are identical.

In the above method, executing the user-centric mutual authentication protocol is initiated only after the sensor system receives multiple haptic taps. In the above method, executing the user-centric mutual authentication protocol is initiated only when at least one of the sensor system or the display device is in a designated location. The above method further comprises: hashing a first serial identification number (SIN) of the sensor system using a SIN hashing algorithm; transmitting a hash of the first SIN to the display device, wherein the display device: uses the SIN hashing algorithm to hash a second SIN obtained by the display device through scanning a quick response (QR) code on the sensor system; compares the hash of the first SIN received from the sensor system with a hash of the second SIN generated through hashing the second SIN obtained by the display device through scanning the QR code; and confirms an identify of the sensor system upon determining that the hash of the first SIN and the hash of the second SIN are identical.

### BRIEF DESCRIPTION OF THE DRAWINGS

FIG. 1A illustrates an example diabetes management system, according to some embodiments disclosed herein.

FIG. 1B illustrates the example diabetes management system of FIG. 1A in more detail, according to some embodiments disclosed herein.

FIG. 2A is a sequence diagram illustrating example operations performed by a diabetes trust management system and a display device, according to some embodiments disclosed herein.

FIG. 2B is a sequence diagram illustrating example operations performed by a diabetes trust management system and a display device, according to some embodiments disclosed herein.

FIG. 3 is a sequence diagram illustrating an example secure diabetes device identification protocol, according to some embodiments disclosed herein.

FIG. 4A is a sequence diagram illustrating an example device-centric mutual authentication protocol (interchangeably referred to as “device-centric authentication protocol”), according to some embodiments disclosed herein.

FIG. 4B is an example chain of certificates associated with an analyte sensor system (“SS”), according to some embodiments disclosed herein.

FIG. 4C is a sequence diagram illustrating an example device-centric mutual authentication protocol, according to some embodiments disclosed herein.

FIG. 4D is a sequence diagram illustrating an example device-centric mutual authentication protocol, according to some embodiments disclosed herein.

FIG. 5 is a sequence diagram illustrating an example user-centric mutual authentication protocol (interchangeably referred to as “user-centric authentication protocol”), according to some embodiments disclosed herein.

FIG. 6A is a sequence diagram illustrating an example key verification protocol, according to some embodiments disclosed herein.

FIG. 6B is a sequence diagram illustrating an example key verification protocol, according to some embodiments disclosed herein.

FIG. 6C is a sequence diagram illustrating an example key verification protocol, according to some embodiments disclosed herein.

FIG. 7 is a sequence diagram illustrating a display device and a SS engaging in pairing and bonding, according to some embodiments disclosed herein.

FIG. 8 is a sequence diagram illustrating a display device and a SS periodically reconnecting, according to some embodiments disclosed herein.

FIG. 9 is a sequence diagram illustrating a display device (that is configured for scanning a QR code) and a SS engaging in a user-centric mutual authentication protocol followed by a device-centric protocol, according to some embodiments disclosed herein.

FIG. 10 is a sequence diagram illustrating a display device (that is not configured for scanning a QR code) and a SS engaging in a user-centric mutual authentication protocol followed by a device-centric authentication protocol, according to some embodiments disclosed herein.

FIG. 11 illustrates a display device and a SS and/or other entities engaging in a device-centric mutual authentication protocol, according to some embodiments disclosed herein.

FIG. 12 is sequence diagram illustrating a display device and a SS using an authorization key (K-auth) for data encryption and/or verifying data authenticity/integrity, according to some embodiments disclosed herein.

### DETAILED DESCRIPTION OF CERTAIN INVENTIVE EMBODIMENTS

Certain embodiments described herein relate to a number of different security protocols used by a display device, a sensor system, a medical device (e.g., a medical delivery device) and/or a server system to establish secure wireless connections, thereby, reducing safety, integrity, privacy, and/or availability issues associated with wireless communications in a diabetes management system. Note that although certain embodiments herein are described with respect to the management of diabetes, a glucose sensor system, and the transmission of glucose measurement between the devices, the protocols and techniques described herein are similarly applicable to any type of health management system that includes any type of analyte sensor (e.g., lactate sensor, ketone sensor, . . . ).

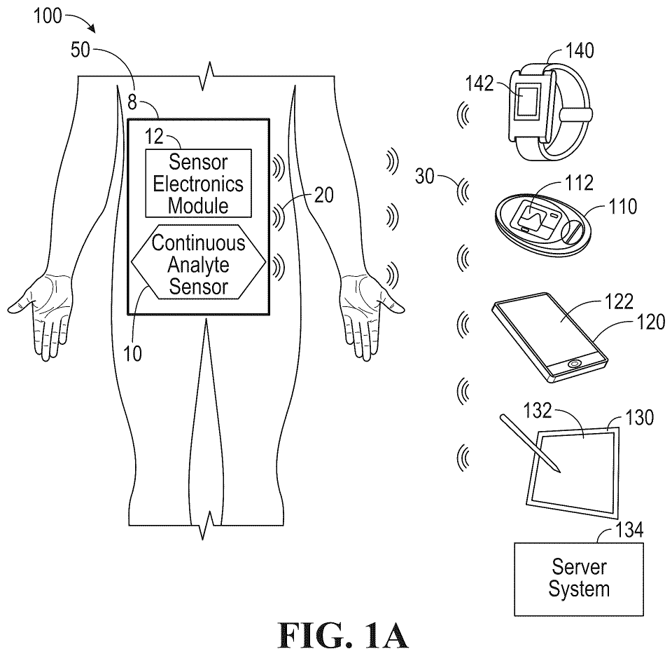

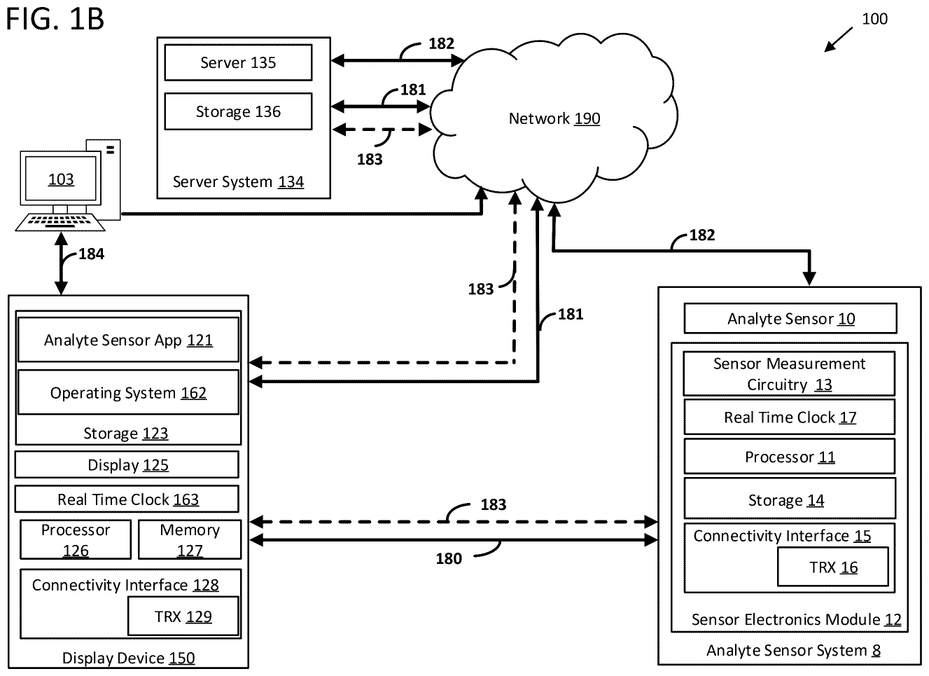

FIG. 1A depicts a disease management system 100 (“system 100”), such as a diabetes management system, that may be used in connection with embodiments of the present disclosure that involve gathering, monitoring, and/or providing information regarding analyte values present in a user's body, including for example the user's blood glucose values. System 100 depicts aspects of analyte sensor system 8 (hereinafter “SS 8”) that may be communicatively coupled to display devices 110, 120, 130, and 140, and/or server system 134.

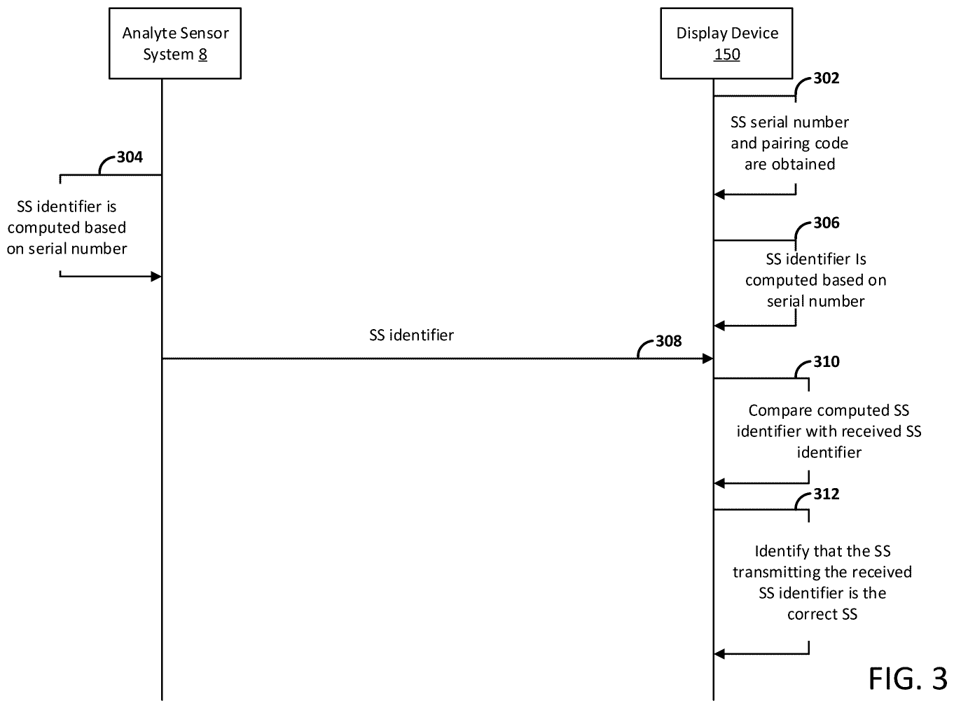

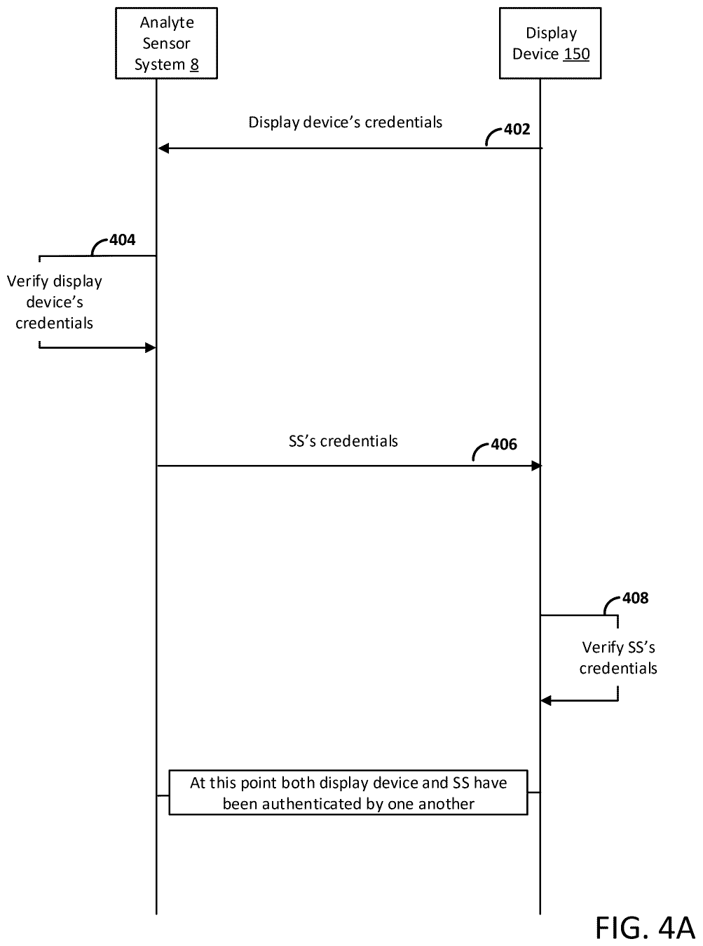

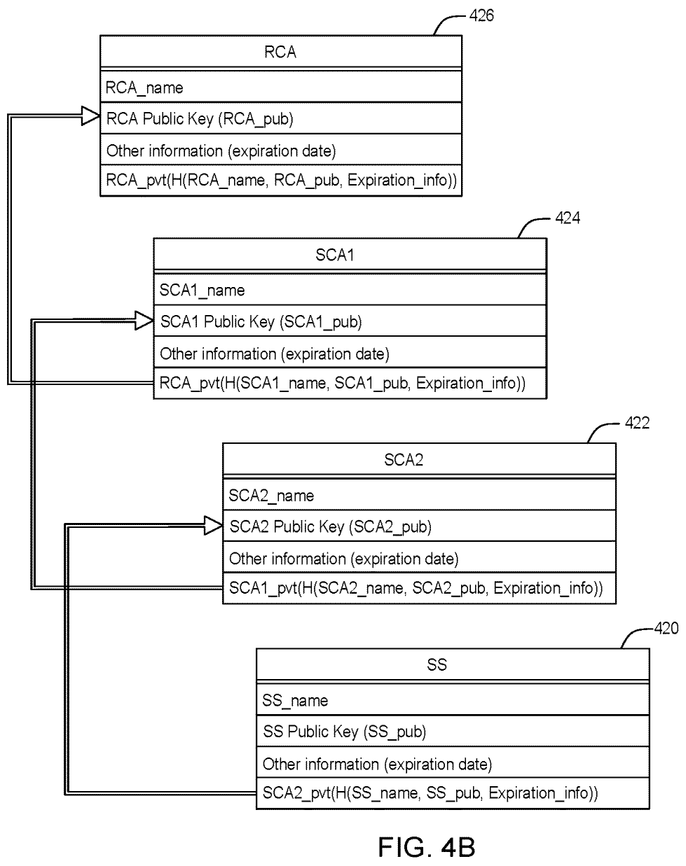

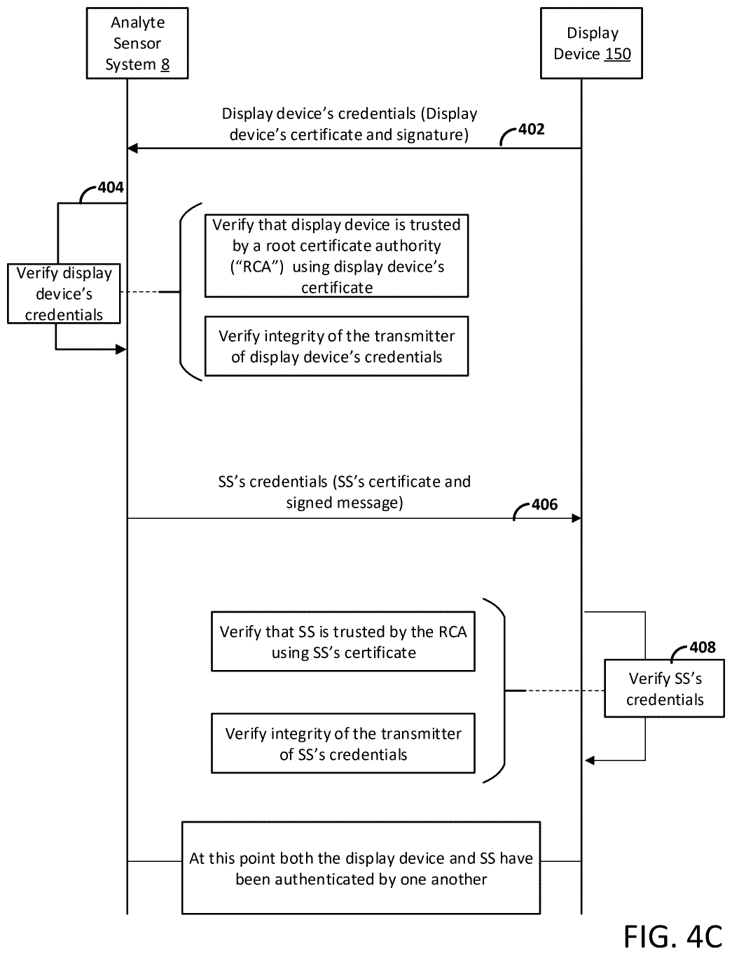

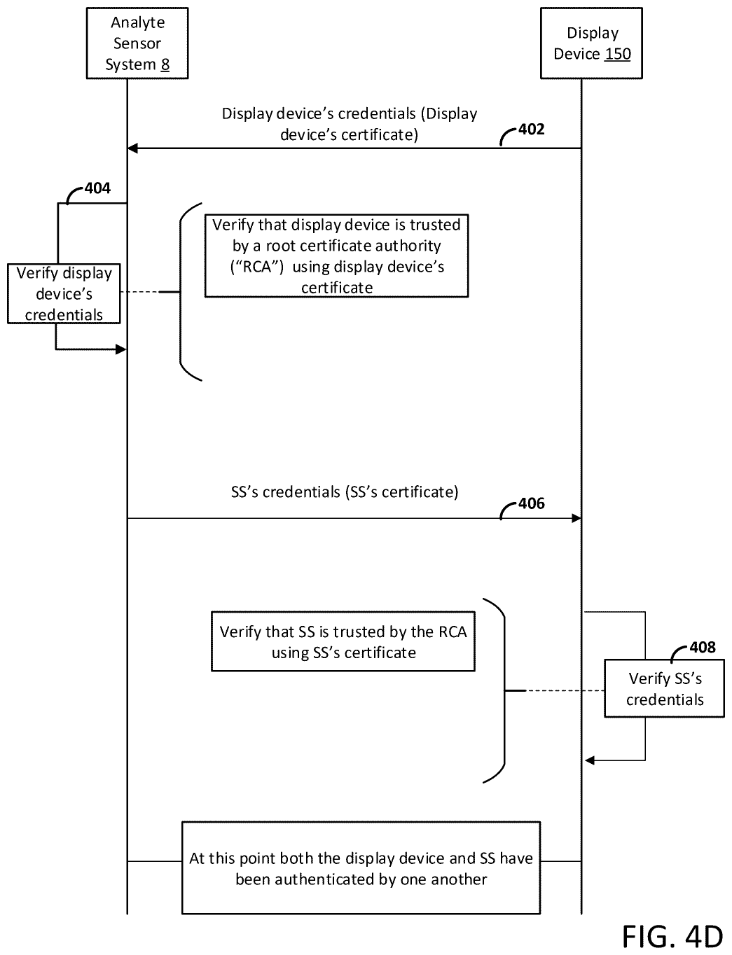

In some embodiments, SS 8 is provided for measurement of an analyte in a host or a user. By way of an overview and an example, SS 8 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data to remote devices, such as display devices 110, 120, 130, 140, and/or server system 134. Paragraphs [0137]-[0140] and FIGS. 3A, 3B, and 4 of U.S. App. No. 2019/0336053 further describe an on-skin sensor assembly that, in certain embodiments, may be used in connection with SS 8. Paragraphs [0137]-[0140] and FIGS. 3A, 3B, and 4 of U.S. App. No. 2019/0336053 are incorporated herein by reference.

In certain embodiments, SS 8 includes an analyte sensor electronics module 12 and an analyte sensor 10 associated with analyte sensor electronics module 12. In certain embodiments, analyte sensor electronics module 12 includes electronic circuitry associated with measuring and processing analyte sensor data or information, including algorithms associated with processing and/or calibration of the analyte sensor data/information. Analyte sensor electronics module 12 may be physically/mechanically connected to analyte sensor 10 and can be integral with (i.e., non-releasably attached to) or releasably attachable to analyte sensor 10.

Analyte sensor electronics module 12 may also be electrically coupled to analyte sensor 10, such that the components may be electromechanically coupled to one another (e.g., (a) prior to insertion into a patient's body, or (b) during the insertion into the patient's body). Analyte sensor electronics module 12 may include hardware, firmware, and/or software that enable measurement and/or estimation of levels of the analyte in a host/user via analyte sensor 10 (e.g., which may be/include a glucose sensor). For example, analyte sensor electronics module 12 can include one or more potentiostats, a power source for providing power to analyte sensor 10, other components useful for signal processing and data storage, and a telemetry module for transmitting data from the sensor electronics module to one or more display devices. Electronics can be affixed to a printed circuit board (PCB) within SS 8, or platform or the like, and can take a variety of forms. For example, the electronics can take the form of an integrated circuit (IC), such as an Application-Specific Integrated Circuit (ASIC), a microcontroller, a processor, and/or a state machine.

Analyte sensor electronics module 12 may include sensor electronics that are configured to process sensor information, such as sensor data, and generate transformed sensor data and displayable sensor information. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

Analyte sensor 10 is configured to measure a concentration or level of the analyte in the host. The term analyte is further defined by paragraph [0117] of U.S. App. No. 2019/0336053. Paragraph [0117] of U.S. App. No. 2019/0336053 is incorporated herein by reference. In some embodiments, analyte sensor 10 comprises a continuous glucose sensor, such as a subcutaneous, transdermal (e.g., transcutaneous), or intravascular device. In some embodiments, analyte sensor 10 can analyze a plurality of intermittent blood samples. Analyte sensor 10 can use any method of glucose-measurement, including enzymatic, chemical, physical, electrochemical, spectrophotometric, polarimetric, calorimetric, iontophoretic, radiometric, immunochemical, and the like. Additional details relating to a continuous glucose sensor are provided in paragraphs [0072]-[0076] of U.S. application Ser. No. 13/827,577. Paragraphs [0072]-[0076] of U.S. application Ser. No. 13/827,577 are incorporated herein by reference.

With further reference to FIG. 1A, display devices 110, 120, 130, and/or 140 can be configured for displaying (and/or alarming) displayable sensor information that may be transmitted by sensor electronics module 12 (e.g., in a customized data package that is transmitted to the display devices based on their respective preferences). Each of display devices 110, 120, 130, or 140 may respectively include a display such as touchscreen display 112, 122, 132, and/or 142 for displaying sensor information and/or analyte data to a user and/or receiving inputs from the user. For example, a graphical user interface (GUI) may be presented to the user for such purposes. In certain embodiments, the display devices may include other types of user interfaces such as voice user interface instead of or in addition to a touchscreen display for communicating sensor information to the user of the display device and/or receiving user inputs. In certain embodiments, one, some, or all of display devices 110, 120, 130, 140 may be configured to display or otherwise communicate the sensor information as it is communicated from sensor electronics module 12 (e.g., in a data package that is transmitted to respective display devices), without any additional prospective processing required for calibration and/or real-time display of the sensor data.

The plurality of display devices 110, 120, 130, 140 depicted in FIG. 1A may include a custom or proprietary display device, for example, analyte display device 110, especially designed for displaying certain types of displayable sensor information associated with analyte data received from sensor electronics module 12 (e.g., a numerical value and/or an arrow, in certain embodiments). In certain embodiments, one of the plurality of display devices 110, 120, 130, 140 includes a smartphone, such as mobile phone 120, based on an Android, iOS, or another operating system configured to display a graphical representation of the continuous sensor data (e.g., including current and/or historic data). In certain embodiments, disease management system 100 further includes a medical delivery device (e.g., an insulin pump or pen). Sensor electronics module 12 may be configured to transmit sensor information and/or analyte data to medical delivery device. The medical delivery device (not shown) may be configured to administer a certain dosage of insulin or another medicament to the user based on the sensor information and/or analyte data (e.g., which may include a recommended insulin dosage) received from the sensor electronics module 12.

Server system 134 may be used to directly or indirectly collect analyte data from SS 8 and/or the plurality of display devices, for example, to perform analytics thereon, generate universal or individualized models for glucose levels and profiles, provide services or feedback, including from individuals or systems remotely monitoring the analyte data, perform or assist SS 8 and display device 150 with identification, authentication, etc., according to the embodiments described herein, so on. Note that, in certain embodiments, server system 134 may be representative of multiple systems or computing devices that perform the functions of server system 134 (e.g., in a distributed manner).

FIG. 1B illustrates a more detailed view of system 100 including a display device 150 that is communicatively coupled to SS 8. In certain embodiments, display device 150 may be any one of display devices 110, 120, 130, and 140 of FIG. 1A. The communication path between SS 8 and display device 150 is shown as communication path 180. In certain embodiments, SS 8 and display device 150 are configured to wirelessly communicate over communication path 180 using low range and/or distance wireless communication protocols. Examples of low range and/or distance wireless communication protocols include Bluetooth and Bluetooth Low Energy (BLE) protocols. In certain embodiments, other short range wireless communications may include Near Field Communications (NFC), radio frequency identification (RFID) communications, IR (infra red) communications, optical communications. In certain embodiments, wireless communication protocols other than low range and/or distance wireless communication protocols may be used for communication path 180, such as WiFi Direct. Display device 150 is also configured to connect to network 190 (e.g., local area network (LAN), wide area network (WAN), the Internet, etc.). For example, display device 150 may connect to network 190 via a wired (e.g., Ethernet) or wireless (e.g., WLAN, wireless WAN, cellular, Mesh network, personal area network (PAN) etc.) interface. Display device 150 is able to communicate with server system 134 through network 190. The communication path between display device 150 and server system 134 is shown as communication path 181 via network 190.

Note that, in certain embodiments, SS 8 may be able to independently (e.g., wirelessly) communicate with server system 134 through network 190. An independent communication path between SS 8 and server system 134 is shown as communication path 182. However, in certain other embodiments, SS 8 may not be configured with the necessary hardware/software to establish, for example, an independent wireless communication path with server system 134 through network 190. In such embodiments, SS 8 may communicate with server system 134 through display device 150. An indirect or pass-through communication path between SS 8 and server system 134 is shown as communication path 183.

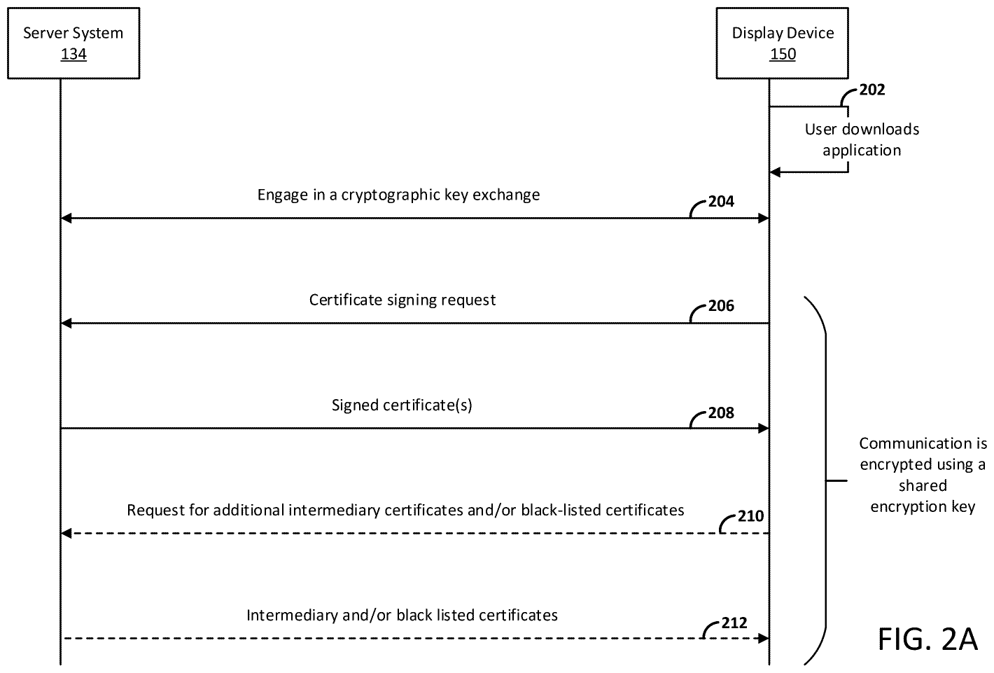

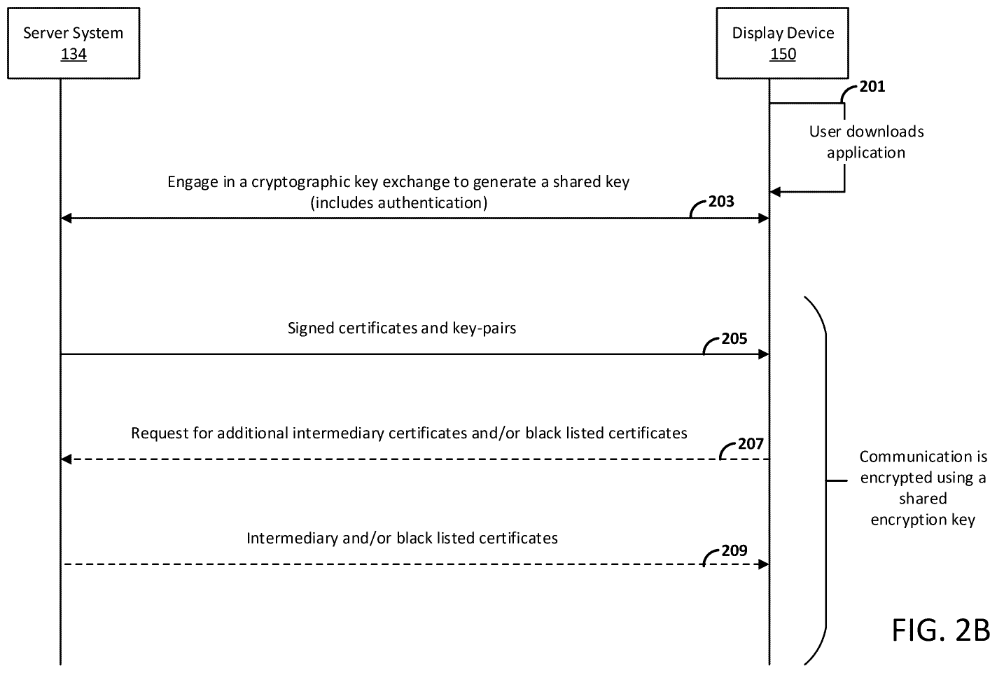

In embodiments where display device 150 is a proprietary display device, such as display device 110 designed specifically for the communication of analyte data, display device 150 may not be configured with the necessary hardware/software for independently connecting to network 190. Instead, in certain such embodiments, display device 150 is configured to establish a wired or wireless communication path 184 (e.g., through a Universal System Bus (USB) connection) with computer device 103, which is configured to communicate with server system 134 through network 190. For example, computer device 103 may connect to network 190 via a wired (e.g., Ethernet) or wireless (e.g., WLAN, wireless WAN, cellular, etc.) interface. Note that in the embodiments described in relation to FIGS. 2A-8, unless otherwise noted, display device 150 is assumed to be capable of independently communicating with server system 134 through network 190, independent of computer device 103.

System 100 additionally includes server system 134, which in turn includes server 135 that is coupled to storage 136 (e.g., one or more computer storage systems, cloud-based storage systems and/or services, etc.). In certain embodiments, server system 134 may be located or execute in a public or private cloud. In certain embodiments, server system 134 is located or executes on-premises (“on-prem”). As discussed, server system 134 is configured to receive, collect, and/or monitor information, including analyte data and related information, as well as encryption/authentication information from SS 8 and/or display device 150. Such information may include input responsive to the analyte data or input (e.g., the user's glucose measurements and other physiological/behavioral information) received in connection with an analyte monitoring or sensor application running on SS 8 or display device 150. This information may be stored in storage 136 and may be processed, such as by an analytics engine capable of performing analytics on the information. An example of an analyte sensor application that may be executable on display device 150 is analyte sensor application 121, as further described below.

In certain embodiments, server system 134 at least partially directs communications between SS 8 and display device 150, for example, for facilitating authentication therebetween. Such communications include messaging (e.g., advertisement, command, or other messaging), message delivery, and analyte data. For example, in certain embodiments, server system 134 may process and exchange messages between SS 8 and display device 150 related to frequency bands, timing of transmissions, security, alarms, and so on. In certain embodiments, server system 134 may also update information stored on SS 8 and/or display device 150. In certain embodiments, server system 134 may send/receive information to/from SS 8 and or display device 150 in real-time or sporadically. Further, in certain embodiments, server system 134 may implement cloud computing capabilities for SS 8 and/or display device 150.

FIG. 1B also illustrates the components of SS 8 in further detail. As shown, in certain embodiments, SS 8 includes analyte sensor 10 coupled to sensor electronics module 12. Sensor electronics module 12 includes sensor measurement circuitry 13 that is coupled to analyte sensor 10 for processing and managing sensor data. Sensor measurement circuitry 13 may also be coupled to processor 11. In some embodiments, processor 11 may perform part or all of the functions of the sensor measurement circuitry 13 for obtaining and processing sensor measurement values from analyte sensor 10. Processor 11 may also be coupled to storage 14 and real time clock (RTC) 17 for storing and tracking sensor data. In addition, processor 11 may be further coupled to a connectivity interface 15, which includes a radio unit or transceiver (TRX) 16 for sending sensor data and receiving requests and commands from an external device, such as display device 150. As used herein, the term transceiver generally refers to a device or a collection of devices that enable SS 8 to (e.g., wirelessly) transmit and receive data. SS 8 may further include storage 14 and real time clock (RTC) 17 for storing and tracking sensor data. It is contemplated that, in some embodiments, the SMC 13 may carry out all the functions of the processor 11 and vice versa.

Transceiver 16 may be configured with the necessary hardware and wireless communications protocols for enabling wireless communications between SS 8 and other devices, such as display device 150 and/or server system 134. For example, as described above, transceiver 16 may be configured with the necessary hardware and communication protocols to establish a Bluetooth or BLE connection with display device 150. As one of ordinary skill in the art appreciates, in such an example, the necessary hardware may include a Bluetooth or BLE security manager and/or other Bluetooth or BLE related hardware/software modules configured for Bluetooth or BLE communications standards. In some embodiments where SS 8 is configured to establish an independent communication path with server system 134, transceiver 16 may be configured with the necessary hardware and communication protocols (e.g., long range wireless cellular communication protocol, such as, GSM, CDMA, LTE, VoLTE, 3G, 4G, 5G communication protocols) for establishing a wireless connection to network 190 to connect with server system 134. As discussed elsewhere, other short range protocols, may also be used for communication between display device 150 and a SS 8 such as NFC, RFID, etc.

FIG. 1B similarly illustrates the components of display device 150 in further detail. As shown, display device 150 includes connectivity interface 128, processor 126, memory 127, a real time clock 163, a display 125 for presenting a graphical user interface (GUI), and a storage 123. A bus (not shown here) may be used to interconnect the various elements of display device 150 and transfer data between these elements. Connectivity interface 128 includes a transceiver (TRX) 129 used for receiving sensor data from SS 8 and for sending requests, instructions, and/or data to SS 8 as well as server system 134. Transceiver 129 is coupled to other elements of display device 150 via connectivity interface 128 and/or the bus. Transceiver 129 may include multiple transceiver modules operable on different wireless standards. For example, transceiver 129 may be configured with one or more communication protocols, such as wireless communication protocol(s) for establishing a wireless communication path with network 190 and/or low range wireless communication protocol(s) (e.g., Bluetooth or BLE) for establishing a wireless communication path 180 with SS 8. Additionally, connectivity interface 128 may in some cases include additional components for controlling radio and/or wired connections, such as baseband and/or Ethernet modems, audio/video codecs, and so on.

In some embodiments, when a standardized communication protocol is used between display device 150 and SS 8, commercially available transceiver circuits may be utilized that incorporate processing circuitry to handle low level data communication functions such as the management of data encoding, transmission frequencies, handshake protocols, security, and the like. In such embodiments, processor 126 of display device 150 and/or processor 11 of SS 8 may not need to manage these activities, but instead provide desired data values for transmission, and manage high level functions such as power up or down, set a rate at which messages are transmitted, and the like. Instructions and data values for performing these high level functions can be provided to the transceiver circuits via a data bus and transfer protocol established by the manufacturer of transceivers 129 and 16. However, in embodiments where a standardized communication protocol is not used between transceivers 129 and 16 (e.g., when non-standardized or modified protocols are used), processors 126 and 11 may be configured to execute instructions associated with proprietary communications protocols (e.g., one or more of the communications protocols described herein) to control and manage their respective transceivers. In addition, when non-standardized or modified protocols are used, customized circuitries may be used to service such protocols.

Processor 126 may include processor sub-modules, including, by way of example, an applications processor that interfaces with and/or controls other elements of display device 150 (e.g., connectivity interface 128, analyte sensor application 121 (hereinafter “sensor application 121”), display 125, RTC 163, memory 127, storage 123, etc.). In certain embodiments, processor 126 is configured to perform functions related to device management, such as, for example, managing lists of available or previously paired devices, information related to network conditions (e.g., link quality and the like), information related to the timing, type, and/or structure of messaging exchanged between SS 8 and display device 150, and so on. Processor 126 may further be configured to receive and process user input, such as, for example, a user's biometric information, such as the user's finger print (e.g., to authorize the user's access to data or to be used for authorization/encryption of data, including analyte data), as well as analyte data.

Processor 126 may include and/or be coupled to circuitry such as logic circuits, memory, a battery and power circuitry, and other circuitry drivers for periphery components and audio components. Processor 126 and any sub-processors thereof may include logic circuits for receiving, processing, and/or storing data received and/or input to display device 150, and data to be transmitted or delivered by display device 150. As described above, processor 126 may be coupled by a bus to display 125, connectivity interface 128, storage 123, etc. Hence, processor 126 may receive and process electrical signals generated by these respective elements and thus perform various functions. By way of example, processor 126 may access stored content from storage 123 and memory 127 at the direction of analyte sensor application 121, and process the stored content to be displayed by display 125. Additionally, processor 126 may process the stored content for transmission via connectivity interface 128 to SS 8 and/or server system 134. Display device 150 may include other peripheral components not shown in detail in FIG. 1B.

In certain embodiments, memory 127 may include volatile memory, such as random access memory (RAM) for storing data and/or instructions for software programs and applications, such as analyte sensor application 121. Display 125 presents a GUI associated with operating system 162 and/or analyte sensor application 121. In various embodiments, a user may interact with analyte sensor application 121 via a corresponding GUI presented on display 125. By way of example, display 125 may be a touchscreen display that accepts touch input. Analyte sensor application 121 may process and/or present analyte-related data received by display device 150 and present such data via display 125. Additionally, analyte sensor application 121 may be used to obtain, access, display, control, and/or interface with analyte data and related messaging and processes associated with SS 8 (e.g., and/or any other medical device (e.g., insulin pump or pen) that are communicatively coupled with display device 150), as is described in further detail herein.

Storage 123 may be a non-volatile storage for storing software programs, instructions, data, etc. For example, storage 123 may store analyte sensor application 121 that, when executed using processor 126, for example, receives input (e.g., by a conventional hard/soft key or a touch screen, voice detection, or other input mechanism), and allows a user to interact with the analyte data and related content via display 125. In various embodiments, storage 123 may also store user input data and/or other data collected by display device 150 (e.g., input from other users gathered via analyte sensor application 121). Storage 123 may further be used to store volumes of analyte data received from SS 8 (or any other medical data received from other medical devices (e.g., insulin pump, pen, etc.) for later retrieval and use, e.g., for determining trends and triggering alerts.

As described above, SS 8, in certain embodiments, gathers analyte data from analyte sensor 10 and transmits the same or a modified version of the collected data to display device 150. Data points regarding analyte values may be gathered and transmitted over the life of analyte sensor 10 (e.g., in the range of 1 to 30 days or more). New measurements may be transmitted often enough to adequately monitor glucose levels. In certain embodiments, rather than having the transmission and receiving circuitry of each of SS 8 and display device 150 continuously communicate, SS 8 and display device 150 may regularly and/or periodically establish a communication channel among each other. Thus, in such embodiments, SS 8 may, for example, communicate with display device 150 at predetermined time intervals. The duration of the predetermined time interval can be selected to be long enough so that SS 8 does not consume too much power by transmitting data more frequently than needed, yet frequent enough to provide substantially real-time sensor information (e.g., measured glucose values or analyte data) to display device 150 for output (e.g., via display 125) to the user. While the predetermined time interval is every five minutes in some embodiments, it is appreciated that this time interval can be varied to be any desired length of time. In other embodiments, transceivers 129 and 16 may be continuously communicating. For example, in certain embodiments, transceivers 129 and 16 may establish a session or connection there between and continue to communicate together until the connection is lost.

Analyte sensor application 121 may be downloaded, installed, and initially configured/setup on display device 150. For example, display device 150 may obtain analyte sensor application 121 from server system 134, or from another source, such as an application store or the like, via a network, e.g., network 190. Following installation and setup, analyte sensor application 121 may be configured to access, process, and/or interface with analyte data (e.g., whether stored on server system 134, locally from storage 123, from SS 8, or any other medical device). By way of example, analyte sensor application 121 may present a menu that includes various controls or commands that may be executed in connection with the operation of SS 8, display device 150, one or more other display devices (e.g., display device 110, 130, 140, etc.), and/or one or more other partner devices, such as an insulin pump. For example, analyte sensor application 121 may be used to interface with or control other display and/or partner devices, for example, to deliver or make available thereto analyte data, including for example by receiving/sending analyte data directly to the other display and/or partner device and/or by sending an instruction for SS 8 and the other display and/or partner device to be connected.

In certain embodiments, after downloading sensor application 121, as one of the initial steps, the user may be directed by sensor application 121 to wirelessly connect display device 150 to the user's SS 8, which the user may have already placed on their body. A wireless communication path 180 between display device 150 and SS 8 allows SS 8 to transmit analyte measurements to display device 150 and for the two devices to engage in any of the other interactions described above. However, as discussed, using a wireless communication path between display device 150 and SS 8, based on certain existing wireless communication protocols, may expose display device 150, SS 8, and/or server system 134 to safety, integrity, privacy, and availability issues. Similarly, establishing other communication paths in system 100 (e.g., communication path 181 between display device 150 and server system 134 as well as communication path 183 between SS 8 and server system 134) using certain existing communication protocols also exposes display device 150, SS 8, and/or server system 134 to safety, integrity, privacy, and availability issues.

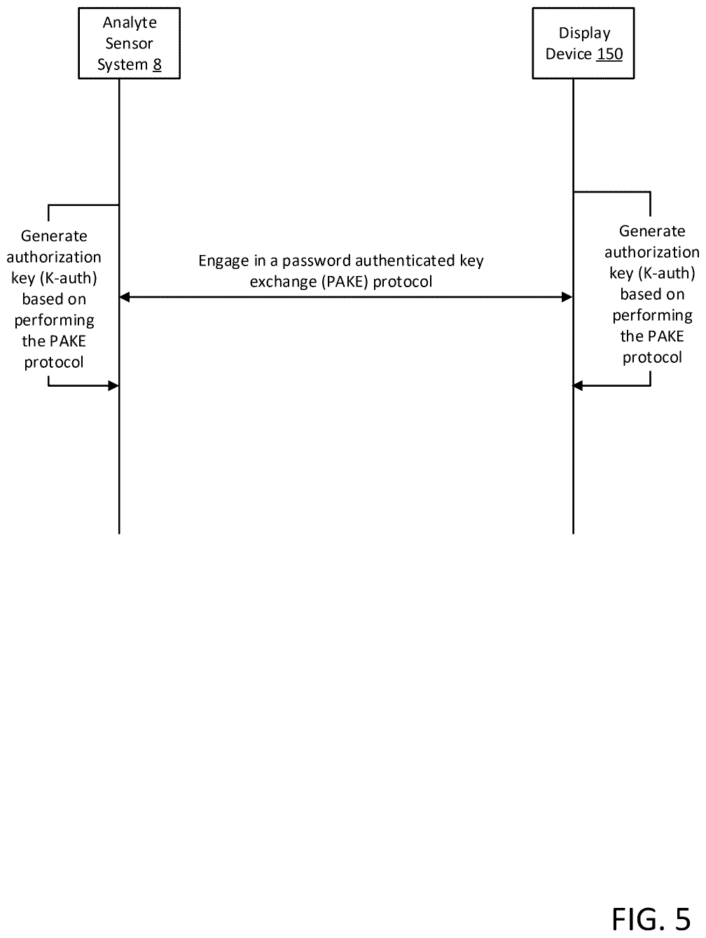

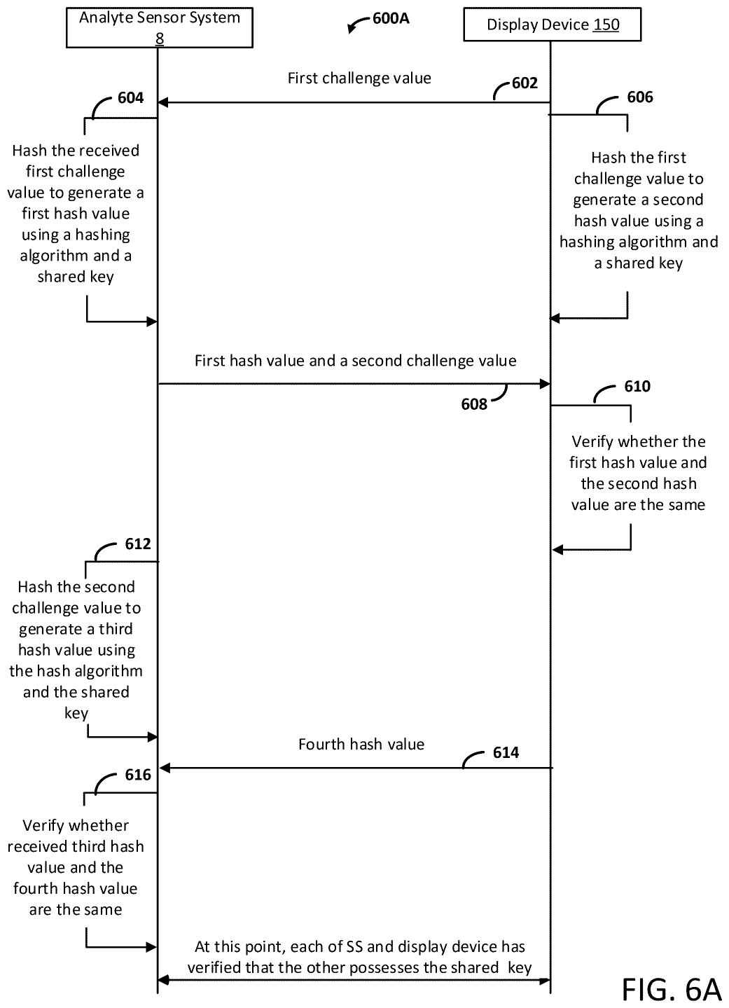

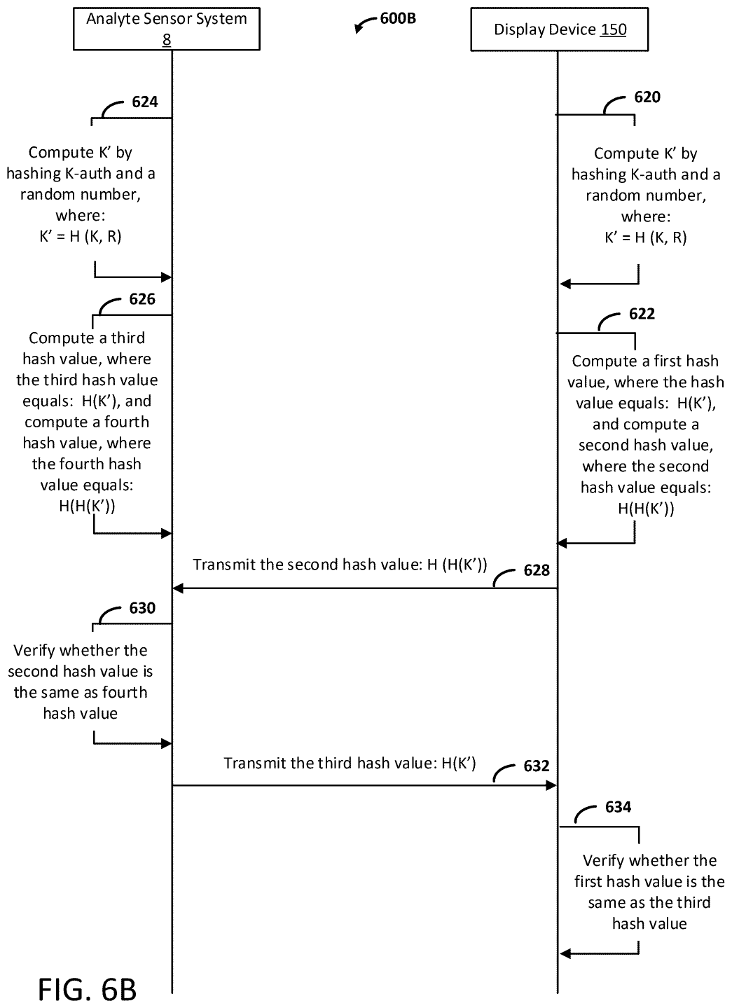

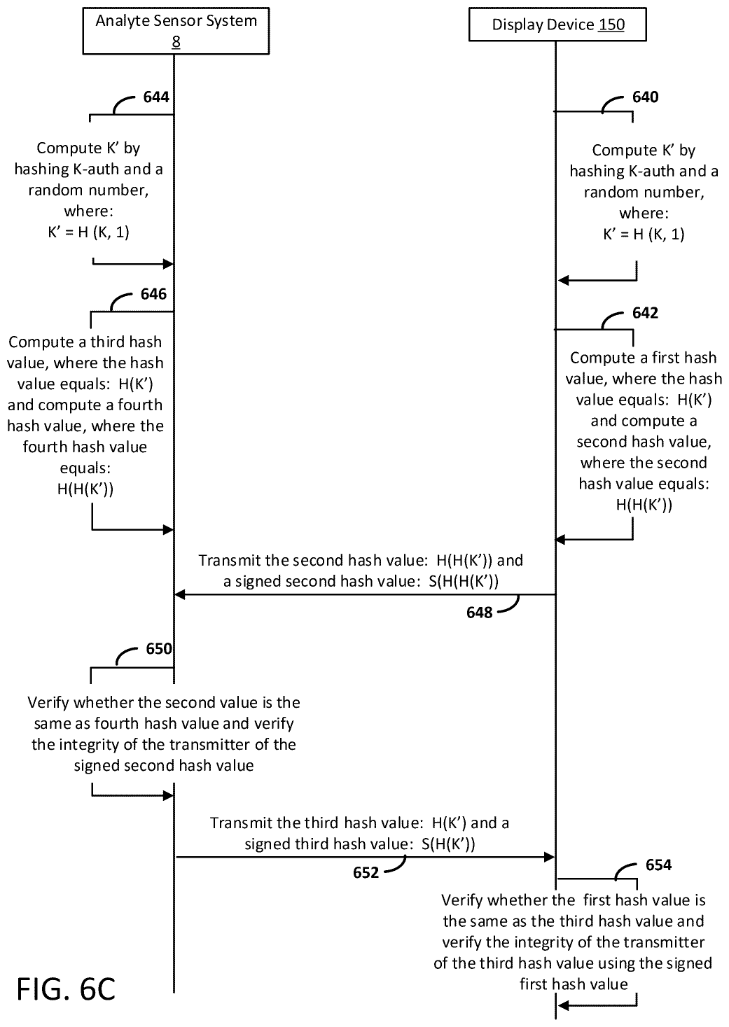

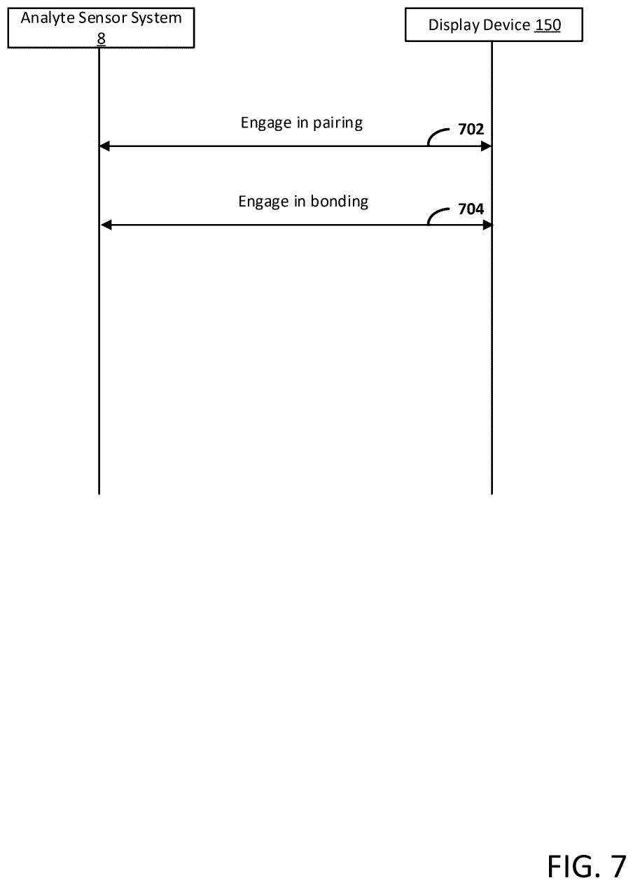

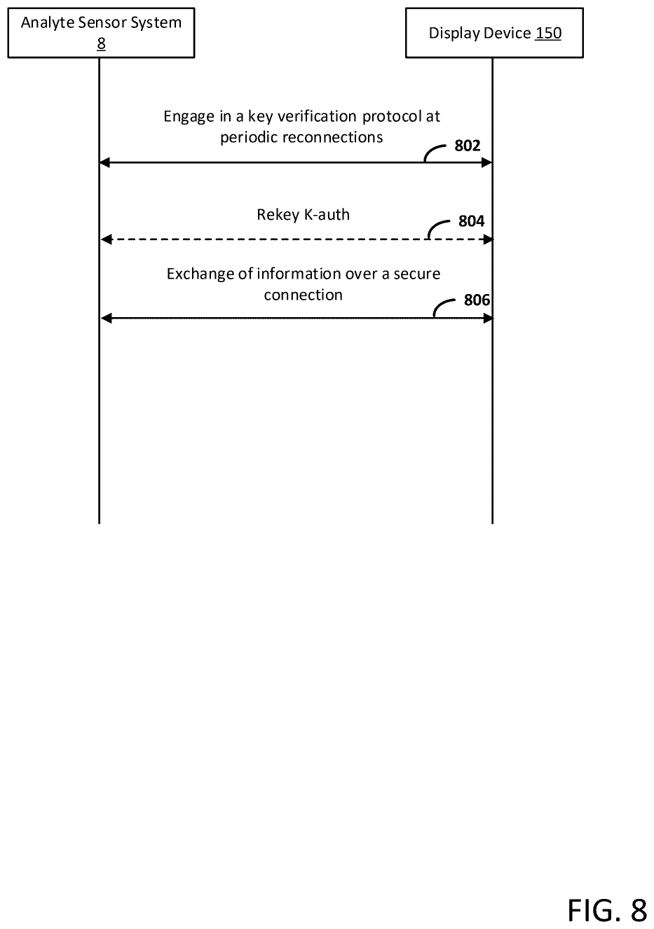

Accordingly, certain embodiments described herein relate to a number of different protocols that may be used to allow display device 150, SS 8, and server system 134 to establish secure communication, thereby, reducing safety, integrity, privacy, and availability issues associated with communications in system 100. FIGS. 2A-4A and 4C-8 are sequence diagrams illustrating communications and exchange of data between server system 134, display device 150, and/or SS 8. More specifically, FIGS. 2A-2B illustrate methods of display device 150 obtaining authentication data (e.g., certificates) from server system 134. FIG. 3 illustrates the execution of an example secure diabetes device identification protocol. FIGS. 4A-4D relate to the execution of example device-centric mutual authentication protocols. FIG. 5 illustrates the execution of an example user-centric mutual authentication protocol. FIGS. 6A-6C relate to the execution of example key verification protocols. FIG. 7 illustrates the execution of pairing and bonding between SS 8 and display device 150. FIG. 8 illustrates an example data exchange between SS 8 and display device during periodic reconnections.

### Secure Data Exchange Between the Display Device and Server System

FIGS. 2A and 2B are sequence diagrams illustrating methods by which display device 150 obtains authentication data from server system 134 during the set-up process of sensor application 121, which executes on display device 150. The secure exchange of data between display device 150 and server system 134, based on any embodiments of any of the methods described with respect to FIGS. 2A-2B, configures sensor application 121 with authentication data that allows display device 150 to subsequently authenticate and establish a secure connection with SS 8, as described in relation to FIGS. 4A-4D. In certain embodiments, the authentication data that sensor application 121 obtains from server system 134 includes a number of digital authentication certificates, also referred to as public key certificates, (herein after “certificates”) for use by display device 150 to authenticate SS 8 using what are referred to herein as device-centric mutual authentication protocols (described in relation to FIGS. 4A-4D). As further described below, SS 8 may similarly be configured with a set of certificates, thereby, allowing SS 8 to authenticate display device 150 using the same device-centric mutual authentication protocols. In certain embodiments, SS 8 is configured with these certificates during the manufacturing process. It is contemplated that in some examples, certificates, tokens, and/or encryption keys may be used for authenticating partner devices (e.g., medical delivery devices, such as insulin pumps and/or pens). It is further contemplated that the certificates, tokens, and/or encryption keys may also be obtained from the partner devices to authenticate SS 8 and/or the display device or other partner devices as well.

In certain embodiments, a certificate is an electronic document generated for authentication of a device that conforms to a public key infrastructure (PKI) scheme. A certificate may be referred to as a credential token herein. PKI refers to a set of roles, policies, hardware, software, and procedures for creating managing, distributing, using, storing, and revoking certificates as well as managing public-key encryption. In a typical PKI scheme, each device may generate or be configured with a key-pair, including a public key and a private key. When information is encrypted using the private key, only the corresponding public key can be used to decrypt that information and vice versa. A public key of the device may be disseminated widely while the device's private key is typically known only to the device and not shared with any other devices. For example, in certain embodiments, each of display device 150, SS 8, and server system 134 may generate or be configured with a distinct key-pair.

As one of its roles, PKI binds public keys with respective identities of devices. The binding is established through a process of registration and issuance of certificates at and by a certificate authority (CA). The primary role of the CA is to digitally sign and publish the public key bound to a given device. This is done using the CA's own private key, so that trust in the user key relies on one's trust in the validity of the CA's key. In certain embodiments, server system 134 performs the functions of a root CA (RCA) by issuing and, directly or indirectly, signing certificates of display device 150 and SS 8. An RCA is an entity that verifies all other entities in a system. As such, server system 134 may be referred to as a diabetes trust management system, which issues and signs certificates of display device 150 and SS 8 or any other partner devices, thereby allowing them to authenticate each other by engaging in the device-centric trust management protocols.

In certain embodiments, server system 134, as the RCA, directly signs a device's certificate (e.g., certificate of display device 150 and/or SS 8) using its private key. Alternatively, in some embodiments, indirect certificate signing may be used, whereby server system 134 signs a subordinate certificate authority's (“SCA's”) intermediary certificate and the SCA then uses its own private key to sign the device's certificate. The involvement of SCAs in certificate signings creates a chain of trust, as shown and described further in relation to FIG. 4B. In certain embodiments, one SCA may be involved while, in certain other embodiments, multiple SCAs may be involved.

The sequence diagrams shown in FIGS. 2A and 2B illustrate communications between display device 150 and server system 134, which result in display device 150 securely obtaining one or more signed certificates from server system 134. Communications between display device 150 and server system 134 may be triggered by the user downloading sensor application 121 or any other application (not shown) associated with the sensor application. As part of the set-up process, sensor application 121 then connects with server system 134 to obtain any necessary information that may be later used for interactions between display device 150 and SS 8.

At step 202, the user downloads sensor application 121. For example, the user downloads sensor application 121 from an application store (e.g., App Store) and initiates the set-up process.

At step 204, display device 150 and server system 134 engage in a cryptographic key exchange. In certain embodiments, during the set-up process, based on instructions provided by analyte sensor application 121, display device 150 engages in or initiates a cryptographic key exchange (e.g., key-agreement protocol such as the Diffie-Hellman (DH) key exchange algorithm) with server system 134 in order to generate an encryption key for use in encrypting any further communications between display device 150 and server system 134. In certain embodiments, engaging in the cryptographic key exchange may involve proving to server system 134 that display device 150 is in possession of a shared secret (e.g., cryptographic key exchange algorithm, key, etc.) and vice versa. Being in possession of the shared secret is proof that display device 150 can be trusted by server system 134 and vice versa. After display device 150 and server system 134 complete the execution of the cryptographic key exchange algorithm, each of display device 150 and server system 134 is in possession of the encryption key that is used to encrypt any subsequent data transmitted there between (e.g., in steps 206-212).

In certain embodiments, the cryptographic key exchange algorithm includes a key-agreement protocol. A key agreement protocol is a protocol whereby two or more parties can agree on a key in such a way that both influence the outcome. One example of a key agreement protocol may be an exponential key exchange algorithm, such as the Diffie-Hellman (DH) key exchange algorithm. DH key exchange is a method of securely exchanging cryptographic keys over an insecure channel. Different versions of the DH key exchange are also within the scope of the disclosure. For example, in certain embodiments, the cryptographic key exchange algorithm may include an elliptic-curve DH (ECDH), which is a key agreement protocol that allows two parties, each having an elliptic-curve public-private key pair, to establish an encryption key over an insecure channel.

In certain embodiments, during the set-up process, based on instructions provided by sensor application 121, display device 150 engages in or initiates a cryptographic key exchange to prove to server system 134 that display device 150 is in possession of a shared secret (e.g., cryptographic key exchange algorithm, etc.). In other words, in certain other embodiments, server system 134 and display device 150 engage in a cryptographic key exchange that does not result in generating an encryption key but merely works to allow server system 134 and display device 150 to authenticate each other. In such embodiments, sensor application 121 is already configured with the shared secret, which may be cryptographic key exchange algorithm. In other words, because display device 150 is able to engage in the cryptographic key exchange algorithm, server system 134 can conclude that display device 150 is trustworthy, otherwise display device 150 would not have been configured with such a cryptographic key exchange algorithm and would not have been able to engage in the cryptographic key exchange using that algorithm.

Once server system 134 is able to determine that display device 150 knows the shared secret, server system 134 determines that it can trust display device 150 and vice versa. In other words, display device 150 is authenticated by server system 134 and vice versa. In certain embodiments, the shared secret is an encryption key that may also be used for encryption of any subsequent data transmitted between server system 134 and display device 150 (e.g., in steps 206-212).

At step 206, display device 150 transmits an encrypted certificate signing request to server system 134. In certain embodiments, display device 150 encrypts the certificate signing request using the encryption key that display device 150 obtained at step 204 or was already configured with. In certain embodiments, display device 150 is configured with a first certificate for use in authentication between display device 150 and SS 8 and a second certificate for use in authentication between display device 150 and server system 134. In certain embodiments, display device 150 is similarly configured with a first key-pair (i.e., a first public key and a first private key) for use in authentication between display device 150 and SS 8 and a second key pair (i.e., a second public key and a second private key) for use in authentication between display device 150 and server system 134.

In certain embodiments, the certificate signing request includes the first certificate and the second certificate. In certain embodiments, the first certificate includes the first public key and the second certificate includes the second public key. The certificate signing request indicates a request to server system 134, which performs the functions of a RCA, for signing the first and the second certificates.

Note that, in certain embodiments, by engaging in the cryptographic key exchange of step 204, display device 150 and server system 134 have already authenticated each other prior to step 206. More specifically, when each device determines that the other is similarly configured with the same cryptographic key exchange algorithm, which may be a custom cryptographic key exchange algorithm, the device may conclude that the other can be trusted. That is because, if the other device was malicious, it would most likely not be configured with the same exact cryptographic key exchange algorithm, or at least a custom cryptographic key exchange algorithm.

However, in certain other embodiments, the second key-pair and the second certificate may additionally or instead be used for authentication in subsequent connections between server system 134 and display device 150. In certain other embodiments, however, additional authentication may not be performed between display device 150 and server system 134 (e.g., to increase resource efficiency, such as compute and storage efficiency). Therefore, display device 150 may not be configured with the second key-pair and a second certificate. In such embodiments, the certificate signing request does not include the second certificate.

At step 208, server system 134 transmits signed certificate(s) to display device 150. As described above, in certain embodiments, server system 134 directly signs certificates and, in certain other embodiments, server system 134 indirectly signs certificates. In certain embodiments, signing a certificate includes encrypting information (or encrypting a hash thereof) included in the certificate, resulting in a digital signature that is then included in the certificate. Examples of signed certificates are shown in FIG. 4B, which is further described below.

In certain embodiments, where the certificate signing request includes the first and the second certificates, server system 134 may sign the certificates using server system 134's private key. In such embodiments, server system 134 may send server system 134's own signed certificate (i.e., signed with server system 134's private key) as well as the first and the second certificates of display device 150, which are signed by server system 134, to display device 150. In certain other embodiments, server system 134 may send server system 134's own signed certificate and display device 150's first and second certificates, which are signed by a SCA, to display device 150. In other words, in such embodiments, the first and the second certificates are not directly signed by server system 134 (e.g., to add an extra layer of security and reduce the risk of any information about the server system 134 or its keys/certificate being exposed). In certain embodiments, the transmission of the one or more signed certificates from server system 134 to display device 150 is encrypted using an encryption key that server system 134 may obtain, generate, or be configured with at step 204.

At optional step 210, display device 150 sends server system 134 a request for intermediary certificates and/or black-listed certificates. For example, if at step 208, server system 134 sends display device 150's certificates that are not directly signed by server system 134, display device 150 may at step 210 then request any intermediary certificates associated with any SCAs involved. In addition, display device 150's request at step 210 may include a request for black-listed certificates. Black-listed certificates refer to certificates of entities (e.g., devices, SCAs, etc.) that should not be trusted by display device 150 any longer. For example, black-listed certificates may indicate unauthorized partner devices (e.g., pump devices) or any unauthorized and/or faulty transmitters.

At optional step 212, server system 134 sends display device 150 any intermediary and/or black-listed certificates that were requested by display device 150 and also available at server system 134. It is contemplated that in some embodiments a server system may be a partner device or a partner server system that the display device communicates for authentication. It is further contemplated that a partner device may communicate with the server system 134 for authentication.

Accordingly, FIG. 2A illustrates a method by which display device 150 transmits a certificate signing request to server system 134, which may include a first and/or a second certificate.

FIG. 2B illustrates an alternative method by which display device 150 obtains authentication data from server system 134. Step 201 is similar to step 202 of FIG. 2A. Steps 203-209 are triggered when step 201 (or a user downloading the application) is performed. In the example of FIG. 2B, display device 150 is not configured with the first key-pair, the second key-pair, a first unsigned certificate, and/or a second unsigned certificate prior to engaging in step 203, which is similar to step 204 of FIG. 2B. Therefore, in such embodiments, at step 205, server system 134 is configured to send display device 150 the first and second signed certificates as well as the first and the second key-pairs. Similar to FIG. 2A, in FIG. 2B, in certain embodiments, the first and/or the second certificates may be directly signed by server system 134 as the RCA. In some such embodiments, at step 205, server system 134 transmits its own signed certificate (i.e., signed with server system 134's private key) as well as the two signed certificates of display device 150 to display device 150. In certain other embodiments, as described above, the first and/or the second certificates are indirectly signed by server system 134, in which case, server system 134 may transmit its own signed certificate and display device 150's first and second certificates signed by a SCA. Steps 207 and 209 are also similar to steps 210 and 212 of FIG. 2A. In step 209, server system 134 transmits signed intermediary certificates of the SCA as well as any other SCAs involved in the chain of trust.

Note that steps 210 and 212 of FIG. 2A as well as steps 207 and 209 of FIG. 2B are optional. In certain embodiments, display device 150 may be configured to obtain any intermediary and/or black-listed certificates from SS 8. In some such embodiments, SS 8 is configured during the manufacturing process with the intermediary and/or black-listed certificates and is further configured to transmit the certificates to display device 150 after all authentications are performed between the two devices. In certain embodiments, SS 8 may only be configured with intermediary certificates during the manufacturing process. As such, in some such embodiments, display device 150 may obtain the intermediary certificates from SS 8 but may still request and obtain the black-listed certificates from server system 134. In certain embodiments where SS 8 is not configured with any necessary intermediary and/or black-listed certificates, display device 150 performs steps 210 and 212 of FIG. 2A or steps 207 and 209 of FIG. 2B, depending on which operations display device 150 and server system 134 engage in.

As described above, in certain embodiments, display device 150 obtains signed certificates and/or any private keys that display device 150 is not already configured with from server system 134 during the set-up process of sensor application 121. Once display device 150 is in possession of this authentication data and the set-up process is complete, display device 150 may be ready to be connected to the user's SS 8. At this point, the user may have also placed SS 8 on their body and ensured that SS 8 is also ready to be connected to display device 150. For display device 150 and SS 8 to connect, however, the display device 150 would first have to identify SS 8 from potentially a number of different SSs in the vicinity. The number of different SSs in the vicinity may include SSs of other users that are close by, such as when the user is in a clinic and there are a group of users close by, all having SSs. In such an example, it is important that display device 150 of the user does not connect to the SS of another user by mistake. The number of different SSs in the vicinity may additionally or instead include a malicious device that is attempting to impersonate the user's SS 8. For example, the attacker may use the malicious device to connect to display device 150 and transmit incorrect data (e.g., incorrect blood glucose measurements) thereto, thereby harming the patient. In addition, as further described below, it is important that display device 150 identifies SS 8 in a secure manner, such that any data exchanged between the two devices for identification purposes, will not be used by an attacker later in the authentication phase.

Accordingly, in certain embodiments, display device 150 and SS 8 engage in one of a set of identification protocols or methods, thereby allowing display device 150 to securely identify the correct SS 8 to connect with. The set of identification protocols may also be referred to as secure diabetes identification protocols.

### Identification

In certain embodiments, an SS is configured with a serial identification (ID) number (hereinafter “SIN” or token ID). In certain cases, this SIN may be indicated (e.g., printed or located) on the SS itself or a package thereof. Once the user obtains this SIN, the user provides the SIN as an input into the sensor application executing on the display device during the set-up process. Having received this SIN, the display device starts searching for the SS with the same SIN, based on a determination that the correct SS to connect with must have the same SIN. Based on certain identification protocols, the SS may be configured to advertise its SIN after the user places the SS on their body and activates the SS. It is noted that, in some cases a SIN may include a modified or a derived version of the SIN. However, advertising the SIN in the clear may, in certain cases, provide an attacker with an opportunity to intercept the advertised SIN during transmission and possibly later use the same SIN to impersonate the SS and authenticate with the display device (depending on the authentication protocols the SS and the display device are configured to perform). Transmitting data in the clear herein refers to transmitting the data in an unencrypted format.

Advertising only a portion of the SIN may also pose issues, especially in cases where SIN is low entropy. Password entropy is a measurement of how unpredictable or identifiable a password is. A low entropy SIN, therefore, is a SIN that is highly identifiable. For example, a low entropy SIN may be six characters long. If an attacker is able to determine, for example, the first two characters of the SIN and the SS advertises the last two characters in the clear during the identification phase, then the attacker may only need to guess the other remaining two characters to authenticate with the display device, if certain existing authentication protocols are used.

Note that, in certain cases, the attacker is able to determine the first two characters because the first two characters of all SSs in the market may be the same. Moreover, during certain authentication protocols that the SS and display device may later engage in, a hash of the SIN may be exchanged between the SS and the display device. In such a case, if the attacker has access to the hash algorithm that was used for hashing the SIN, the attacker is able to identify the two remaining characters that, in combination with the other four characters, would result in the six characters whose hash value is equal to the hash of the SIN that the attacker was able to obtain by eavesdropping during authentication between the SS and the display device.

Accordingly, certain embodiments described herein relate to a number of identification protocols designed to allow display device 150 to effectively identify SS 8 and to reduce or eliminate the likelihood of an attacker being able to obtain any information during the identification process that may become useful in impersonating SS 8 or display device 150.

In certain embodiments, one of a number of proximity-based identification protocols may be used by SS 8 and display device 150. A proximity-based identification protocol helps ensure that only two devices in close proximity to each other are able to communicate and complete the identification process. For example, in certain embodiments, a proximity-based identification protocol may involve configuring a device (e.g., display device 150 and/or SS 8) to only communicate (e.g., for the purpose of identification, authentication, bonding, and/or pairing) with another device with a received signal strength indicator (RSSI) of more than a certain threshold, meaning the device (e.g., display device 150) receives signals (e.g., advertising the SIN), from the other device (e.g., SS 8), that have a signal strength, as measured by the device, that satisfy the threshold. Utilizing RSSI in a proximity-based identification protocol helps ensure that, for example, during the identification phase, display device 150 only identifies SS 8 if SS 8 has an RSSI that is higher than a minimum threshold. Therefore, any SSs with RSSIs lower than the threshold would not be included in display device 150's search. Note that it is likely that a malicious device attempting to impersonate SS 8 will not be very close to the user or at least as close to display device 150 as SS 8. As such, a malicious device with an RSSI lower than the threshold would be excluded from display device 150's search.

In certain embodiments, RSSI may be utilized by a proximity-based identification protocol to generate a signature that can be used for identification. For example, display device 150 may instruct the user to move SS 8 closer and farther, and then closer and farther way from display device 150. This change in the RSSI generates or communicates a unique signature that display device 150 already stores. When the two signatures match, display device 150 is able to conclude that SS 8 is the right SS to connect with.

In certain embodiments, a proximity-based identification protocol may involve configuring display device 150 and SS 8 to reduce their transmission power (e.g., signal transmission power of their antennas) during the identification phase. Using this approach helps ensure that display device 150 will not identify SS 8 unless they are within a relatively close proximity with respect to each other. In certain embodiments, a proximity-based identification protocol may involve the use of both RSSI and transmission power, for example, based on one or more approaches described above. Details regarding protocols involving the use of RSSI are further described in U.S. application Ser. No. 15/782,702, which is incorporated herein by reference in its entirety.

In certain embodiments, a visual-based identification protocol may be used for identification. For example, in certain embodiments, display device 150 may be configured to scan or read an identifier associated with SS 8. For example, an identifier associated with SS 8 may include a SIN written in a visible manner or a SIN that is located or printed on SS 8 or a package thereof using non-visible (to humans) ink, a QR code, and/or symbol(s).

In certain embodiments, an auditory-based identification protocol may be used for identification. For example, SS 8 may be configured to audibly play a sound that the user could identify. Once the user identifies that sound, they are able to determine that display device 150 has identified the correct SS. In certain embodiments, a vibration-based identification protocol may be used for identification. For example, SS 8 may be configured to vibrate in a certain way that would alert the user as to whether or not display device 150 has identified the correct SS.

In certain embodiments, the SIN may be hashed, truncated, or a combination of the two during the identification phase. FIG. 3 illustrates SS 8 and display device 150 performing an example identification protocol that is based on hashing, truncating, and/or a combination of both.

At step 302, display device 150 obtains the SIN as well as a pairing code (e.g., a Bluetooth pairing code). In certain embodiments, the user may input the SIN and the pairing code of SS 8 into a user interface of sensor application 121, executing on display device 150. In certain embodiments, display device may obtain the SIN and the pairing code, which may be located on SS 8 thereof, by scanning them. In certain embodiments, a pairing code refers to a Bluetooth pairing code that may be used later for authentication between display device 150 and SS 8 as well as the actual pairing between the two devices. In certain embodiments, instead of a pairing code, another code or password may be used for the purposes described herein (e.g., with respect to FIG. 5). In certain embodiments, the SIN may be a high entropy SIN. For example, SIN may be 14+/−2 characters long, where each character has, for example, 32 possible values. In the embodiments of FIG. 3, both SS 8 and display device 150 are configured with an algorithm to compute a SS identifier based on the SIN as further discussed below.

At step 304, SS 8 computes an SS identifier based on SIN, using the algorithm. In certain embodiments, the algorithm for computing the SS identifier may be a hashing algorithm that hashes the SIN to a hash value, e.g., the SS identifier. In certain embodiments, the algorithm for computing the SS identifier may be a truncating algorithm that truncates the SIN to generate the SS identifier. In such embodiments, the SS identifier has fewer characters than the SIN. For example, the truncating algorithm may truncate the SIN from 14 characters to 5 characters. In certain embodiments, instead of using a truncating algorithm, the user may be asked to input only a portion of the SIN into display device 150's user interface. In other words, in such embodiments, the user manually truncates the SIN. In such embodiments, the portion of the SIN inputted by the user is used as the SS identifier.

In certain embodiments, the algorithm for computing the SS identifier may use a combination of hashing and truncating. For example, in certain embodiments, SS 8 may first use a hashing algorithm to hash the SIN to a hash value and then use a truncating algorithm to take the hash value as input, and output a truncated version of the hash value (e.g., the last two characters of the hash value). For example, in certain embodiments, when an algorithm that uses both hashing and truncating is used, the computed SS identifier may be as follows:

SS Identifier=“Manufacturer's Name”+Last_N_Chars(Hash Function(SIN))

In the formula above, Last_N_Chars refers to a truncating algorithm that takes an input (e.g., Hash Function(SIN)) and truncates the input to its last N characters, where N may be configured to be any number more than “1.” In certain embodiments, Manufacturer's Name is an optional parameter. Manufacturer's name may be the name of the manufacturer of SS and may appear in letters. Hash Function( ) refers to a hashing algorithm that takes SIN as input and outputs a hash value. In certain other embodiments, the computed SS identifier may be as follows:

SS Identifier=“Manufacturer's Name”+Hash Function(Last_N_Chars(SIN))

After computing the SS identifier, SS 8 is ready to start advertising the SS identifier (e.g., periodically).

At step 306, display device 150 similarly computes the SS identifier based on the SIN, using the same algorithm used by SS 8 at step 304. After computing the SS identifier, display device 150 is ready to start searching for the SS identifier.

At step 308, SS 8 advertises the SS identifier. In certain embodiments, SS 8 starts to periodically advertise the SS identifier immediately after computing it.

At step 310, display device 150 compares the SS identifier computed by display device 150 at step 306 with the SS identifier advertised by SS 8 at step 308.

At step 312, display device 150 identifies that SS 8, which transmitted the SS identifier at step 308, is the correct SS to connect with.

Note that, in certain embodiments, a combination of some of the identification protocols described above may be used. For example, display device 150 and SS 8 may be configured to engage in the identification phase using an algorithm that uses a combination of hashing and truncating while at the same time display device 150 and SS 8 may be further configured with one of the proximity-based techniques described above.

The identification protocols described herein help protect SS 8's SIN, which may be considered personally identifiable information (PII) and/or personal health information (PHI). The identification protocols described herein, thereby, reduce the likelihood of an attacker obtaining any information, during the identification phase, that the attacker can use, for example, to impersonate SS 8 for the purpose of subsequently authenticating and connecting with display device 150. In certain embodiments, display device 150 may not use an in-band or out-of-band identification protocol to identify the correct SS. In such embodiments, display device 150 may connect to any SSs within range until the correct SS is identified.

### Authentication

In certain cases, once a display device identifies a SS, the two devices may be configured to authenticate each other. For example, based on certain authentication protocols, the display device may engage in a challenge-response mechanism with the SS to verify that the SS is in possession of a shared secret, and vice versa. In certain cases, the shared secret may be the SIN. For example, in certain cases, the display device may transmit to the SS a hashed-version of the SIN, such as H(H(SIN)). The SS which is also configured with the same hashing algorithm is similarly able to compute H(H(SIN)) and compare H(H(SIN)) with what was received from the display device. If what was received and what was computed are the same, then SS determines that the display device is in possession of the SIN. Subsequently, the SS sends H(SIN) to the display device, based on which the display device determines that the transmitter is also in possession of the SIN. Once this challenge response mechanism is complete, the SS and the display device determine that they can trust each other.

However, as described above, an attacker that is eavesdropping during this challenge response mechanism may obtain H(SIN) and then try and determine what the SIN is. Determining SIN using H(SIN) in such situations may be possible because the attacker may be in possession of the hash function (e.g., by hacking another SS or display device) and also already know most characters of the SIN. The attacker may know most characters of the SIN based on SINs of other SSs in the market as well as by eavesdropping during a previously described identification procedure. As described above, during certain identification protocols, the SS transmits certain characters of the SIN in the clear. Once an attacker obtains the SIN, the attacker is able to impersonate the SS and authenticate with and connect to the display device by using the SIN. The attacker may also impersonate the display device to connect with the SS using the SIN.

Using the challenge response mechanism (e.g., a type of authentication protocol) described above may further expose the system to risks associated with improper use or access to the SS, display device, and/or a server system that is in communication with the SS and the display device. For example, in certain cases, instead of using a safe SS that is compatible with the sensor application running on the display device, a patient may use a potentially unsafe third party sensor to connect with a trusted sensor application that may or may not be compatible with the third party sensor. In such an example, the third party sensor may also generate inaccurate data, which may be stored in a storage provided by the server. In another example, a patient may use a potentially unsafe sensor application to gain access to the analytics or services provided by the server.

Accordingly, certain embodiments described herein relate to a number of authentication protocols that SS 8 and display device 150 may engage in, after the identification phase, to address the issues described above. More specifically, SS 8 and display device 150 may be configured to perform one of a number of device-centric mutual authentication protocols and/or a number of user-centric mutual authentication protocols. A device-centric mutual authentication protocol, as described in relation to FIGS. 4A-4D, allows display device 150 to verify whether SS 8 is trusted by a RCA, e.g., server system 134, and vice versa. Engaging in a device-centric mutual authentication protocol helps, for example, prevent an untrusted device from gaining access to SS 8 or display device 150, even if the untrusted device is able to obtain SS 8's SIN or another shared secret (e.g., the pairing code).

A user-centric mutual authentication protocol, as described in relation to FIG. 5, allows each of display device 150 and SS 8 to generate an authorization key (“K-auth”) based on a shared secret (e.g., pairing code). In certain embodiments, K-auth is an advanced encryption standard (AES) key. If both display device 150 and SS 8 generate the same K-auth, which is subsequently verified using a key verification protocol, display device 150 and SS 8 are able to conclude that the other is in possession of the shared secret and, therefore, trusted by the user. Note that, as described above, when a user, for example, purchases a trusted SS 8 and downloads a trusted sensor application 121, the user inputs the SIN and a pairing code of SS 8 (or any other type of secret information) into a user interface of sensor application 121. That is because the user trusts both SS 8 and display device 150. If the user does not trust, for example, display device 150 or sensor application 121, the user would not share SS 8's SIN and pairing code with display device 150. In certain embodiments, the user sharing SS 8's SIN and pairing code with display device 150 includes or refers to the user inputting SS 8's SIN and pairing code into a user interface of display device 150. Therefore, by using the user-centric authentication protocol, SS 8 is able to verify whether the patient trusts display device 150 based on whether display device 150 is in possession of the shared secret. In certain embodiments, the pairing code is used as the shared secret. In certain other embodiments, any other identifier associated with SS 8 may be used as the shared secret instead.

In certain embodiments, after the identification phase, display device 150 and SS 8 first perform the device-centric mutual authentication protocol and then the user-centric mutual authentication protocol. In certain other embodiments, after the identification phase, display device 150 and SS 8 first perform the user-centric mutual authentication protocol and then the device-centric authentication protocol. In certain embodiments, after the identification phase, display device 150 and SS 8 may only perform one of the user-centric and device-centric mutual authentication protocols.

### Device-Centric Authenticating Protocol

FIG. 4A is a sequence diagram illustrating display device 150 and SS 8 engaging in a device-centric authentication protocol. FIG. 4B illustrates an example chain of certificates that may be used as part of the device-centric authentication protocol. FIGS. 4C and 4D illustrate alternative and/or additional embodiments for performing the device-centric authentication protocol. As such, FIGS. 4A, 4B, 4C, and 4D are described together for clarity.

At step 402 of FIG. 4A, display device 150 transmits its credentials or authentication data to SS 8. In certain embodiments, as shown in step 402 of FIG. 4C, display device 150's credentials include display device 150's certificate as well as a message signed by display device 150. In certain embodiments, as shown in step 402 FIG. 4D, display device 150's credentials may include display device 150's certificate. In certain embodiments, display device 150's certificate comprises display device 150's public key (“DD-Pub”) and/or additional information, such as the name of display device 150, the certificate's expiration information, etc. In certain embodiments, display device 150's certificate also includes a digital signature of server system 134 or a SCA.

In certain embodiments, a message that is signed by display device 150 refers to a message that includes a first portion that is unencrypted as well as a second portion that includes a digital signature. In certain embodiments, the digital signature refer to an encrypted hash of the first portion. In certain embodiments, display device 150 encrypts the hash of the first portion using its private key (“DD-Priv”).

In certain embodiments where display device 150's certificate is not directly signed by server system 134's private key, display device 150 may also be configured to transmit intermediary certificates of any SCAs involved in display device 150's chain of certificates. However, in certain embodiments, SS 8 may be already configured with such intermediary certificate(s), in which case display device 150 may refrain from transmitting such intermediary certificate(s) to SS 8.

At step 404 of FIG. 4A, SS 8 verifies display device 150's credentials. In certain embodiments, SS 8 is configured (e.g., stores) with a signed certificate of server system 134, including server system 134's public key (“RCA-Pub”). In certain embodiments where display device 150's certificate is signed by server system 134's private key (“RCA-Priv”), SS 8 uses the RCA-Pub to decrypt or verify the digital signature included in display device 150's certificate. Note that the RCA-Pub is able to decrypt what has been signed with the RCA-Priv. Therefore, if SS 8 is able to decrypt the digital signature, and the resulting information is equal to the hash of the first unencrypted portion in display device 150's certificate, then SS 8 is able to conclude that display device 150 is trusted by server system 134 because RCA-Priv was used to sign display device 150's certificate. If SS 8 fails to decrypt the digital signature, SS 8 concludes that display device 150 should not be trusted and ends the device-centric mutual authentication protocol. Note that, in certain embodiments, SS 8 may communicate with and be authenticated by the server system 134 directly and vice versa (via WiFi, cellular, etc.).

In certain embodiments where one or more intermediary certificates are involved in display device 150's chain of certificates, SS 8 authenticates each of the intermediary certificates involved, starting from the intermediary certificate that is signed by RCA-Priv (i.e., the intermediary certificate of the SCA just below server system 134 in the chain of certificates) and ending with the intermediary certificate whose private key was used to sign display device 150's certificate. Once all intermediary certificates are authenticated, SS 8 authenticates display device 150's certificate. Details relating to authenticating a chain of certificates is described with respect to step 408 as well as FIG. 4B, which illustrates SS 8's chain of certificates.

As described above, FIGS. 4C and 4D illustrate alternative embodiments for performing the device-centric authentication protocol, including step 404, shown in FIG. 4A. In certain embodiments, in FIG. 4C, where display device 150's credentials include a message signed by display device 150, at step 404, SS 8 uses the signed message to determine whether or not it was actually display device 150 itself that transmitted display device 150's credentials at step 402. In other words, SS 8 verifies the integrity of the transmitting entity of display device 150's credentials, which involves verifying that the transmitter is in possession of DD-Priv. Note that the transmitting entity of display device 150's credentials is the entity that transmitted the display device 150's credentials. Because no other device than display device 150 can be in possession of DD-Priv, by ensuring that the transmitting entity is in possession of DD-priv, SS 8 may conclude that the transmitting entity of display device 150's credentials is in fact display device 150.

To verify whether the transmitting entity is in possession of DD-Priv, SS 8 uses DD-Pub from display device 150's certificate (which SS 8 already authenticated in step 404) and decrypts the encrypted portion (i.e., second portion) of the signed message. If an unencrypted version of the second portion is equal to a hash of the first portion of the signed message, then it means that the signed message was signed by DD-Priv. Therefore, SS 8 concludes that the transmitting entity that transmitted display device 150's credentials was in fact display device 150 itself. Performing this integrity verification may be advantageous to protect against an attacker that may have improperly obtained display device 150's certificate (e.g., a man in the middle attacker that has intercepted display device 150 during a transmission of display device 150's credentials) and be attempting to impersonate display device 150.

In certain embodiments of FIG. 4D, where display device 150's credentials do not include a message signed by display device 150, the integrity of the transmitting entity of display device 150's credentials may be verified during the execution of a key verification protocol, such as described in relation to FIG. 6C.

Referring back to FIG. 4A, at step 406, SS 8 transmits its credentials to display device 150. In certain embodiments, as shown in step 406 of FIG. 4C, SS 8's credentials include SS 8's certificate as well as a message signed by SS 8. In certain embodiments, as shown in step 406 of FIG. 4D, SS 8's credentials only include SS 8's certificate. In certain embodiments, SS 8's certificate is signed directly by server system 134 and, in certain other embodiments, SS 8's certificate is signed by a SCA. In certain embodiments where SS 8's certificate is signed by a SCA, at step 406, SS 8 may also transmit any intermediary certificates in SS 8's chain of certificates. Note that steps 404-408 are triggered because display device 105 sent its credentials to SS 8 at step 402.

Moving to FIG. 4B, FIG. 4B illustrates a chain of certificates associated with SS 8. In the example of FIG. 4B, two SCAs (e.g., which could be or include one or more servers or computing devices), having intermediary certificates 422 and 424, are involved in SS 8's chain of certificates. As shown, SS 8's chain of certificates starts with SS 8's own certificate 420 and ends with server system 134's certificate 426. In certain embodiments, SS 8's certificate comprises SS 8's public key (“SS-Pub”) and/or additional information, such as the name of SS 8, the certificate's expiration information, etc. SS 8's certificate also includes a digital signature, which refers to a hash of the information in SS 8's certificate 420 (e.g., SS 8's name, SS-Pub, and the certificate's expiration information) that is encrypted with a private key of SCA 2 (e.g., which could be or include one or more servers or computing devices). Similarly, intermediary certificate 422 is signed by a private key of SCA 1 and intermediate certificate 424 is signed by the RCA-Priv of server system 134.

Referring back to FIG. 4A, at step 408, display device 150 verifies SS 8's credentials. For example, display device 150 may initially verify the identity of certificate 424 by decrypting the digital signature in certificate 424 using the RCA-Pub. Following that, display device 150 hashes the information included in certificate 424 (except for the digital signature). If the result of the hashing and the decrypting are the same, the authenticity of certificate 424 is verified. Display device 150 similarly verifies the authenticity of certificates 422 and 420. Once certificate 420 is also verified, display device 150 is able to conclude that SS 8 is trusted by server system 134.

In certain embodiments of FIG. 4C, where SS 8's credentials include a message signed by SS 8, display device 150 uses the signed message to determine whether or not it was actually SS 8 itself that transmitted SS 8's credentials at step 406. Verifying the integrity of the transmitting entity of SS 8's credentials is performed in a similar manner described above. More specifically, verifying the integrity of the transmitting entity of SS 8's credentials includes decrypting the signed message using SS-Pub and hashing the information in the message. If the result of the decryption and hashing are the same, then display device 150 concludes that the transmitting entity of SS 8's credentials was in fact SS 8 itself.

Once display device 150 and SS 8 have verified each other's credentials, they have each authenticated that the other is trusted by server system 134, e.g., the diabetes trust management system.

FIG. 4C, as described above, illustrates a device-centric authentication protocol whereby display device 150 and SS 8 are configured to transmit signed messages to each other for integrity verification purposes. FIG. 4D, as described above, illustrates a device-centric authentication protocol whereby display device 150 and SS 8 are configured not to transmit signed messages to each other for integrity verification purposes.

### User-Centric Authentication Protocol

FIG. 5 illustrates display device 150 and SS 8 engaging in a user-centric mutual authentication protocol. As described above, by engaging in the user-centric mutual authentication protocol, each of display device 150 and SS 8 is able to generate an authentication key (e.g., K-auth), based on a shared secret (e.g., pairing code). If both display device 150 and SS 8 generate the same K-auth, which is subsequently verified using a key verification protocol, each of display device 150 and SS 8 is able to conclude that the other is in possession of the shared secret and, therefore, trusted by the user. The user-centric authentication protocol may include one of a variety of password authenticated key exchange (PAKE) algorithms. PAKE is a cryptographic key exchange protocol. A distinguishing feature of PAKE is that one device (e.g., SS 8) is able to authenticate itself to the other device (e.g., display device 150) using a password or shared secret (e.g., pairing code). PAKE is further designed to allow two parties (e.g., display device 150 and SS 8) to generate or derive a high entropy authentication key (e.g., K-auth) from a shared low entropy secret (e.g., pairing code). If an attacker engages in a PAKE protocol with a device, the attacker is only able to make a single guess of the shared secret and, further, will only learn that its guess was incorrect. No other information is leaked.

One of a variety of PAKE algorithms may be used as part of the user-centric authentication protocol. Examples include Juggling PAKE or J-PAKE, EC-J-PAKE (elliptic curve cryptography), SPEKE (simple password exponential key exchange), CRS-J-PAKE (common reference string-J-PAKE), AuCPace (Augmented Composable Password Authenticated Connection Establishment), BSPEKE (a “B” extension for SPEKE), zkPAKE (zero-knowledge PAKE), C2C-PAKE (client to client PAKE), and EKE (encrypted key exchange).

An example of how the J-PAKE algorithm may be performed between two devices is described on page 10 of F. Hao, P. Ryan. J-PAKE: Authenticated Key Exchange Without PKI, Springer Transactions on Computational Science XI, Special Issue on Security in Computing, Part II, Vol. 6480, pp. 192-206, 2010 (hereinafter “Hao”).

As described on page 10 of Hao, G denotes a subgroup of Z*r with prime order q in which the Decision Diffie-Hellman problem (DDH) is intractable. g is a generator in G. The two communicating parties, SS 8 and display device 150, both agree on (G, g). s (e.g., pairing code) is their shared password, and s≠0 for any non-empty password. The value of s falls within [1, q−1]. Display device 150 selects two secret values x1 and x2 at random: x1∈_{R}[0, q−1] and x2 ∈_{R} [1, q−1]. Similarly, SS 8 selects x3∈_{R} [0, q−1] and x4∈_{R} [1, q−1]. Note that x2, x4≠0.

In Round 1 of J-PAKE, display device 150 sends out g^{x1}, g^{x2} and knowledge proofs for x1 and x2. Similarly, SS 8 sends out g^{x3}, g^{x4} and knowledge proofs for x3 and x4. The above communication can be completed in one round as neither party depends on the other. When Round 1 finishes, SS 8 and display device 150 verify the received knowledge proofs, and also check g^{x2}, gₓ₄≠1.

In Round 2 of J-PAKE, display device 150 sends out DD=g^{(x1+x3+x4)·x2·s} and a knowledge proof for x2·s. Similarly, SS 8 sends out SS=g^{(x1+x2+x3)·x4·s} and a knowledge proof for x4·s. When this round finishes, display device 150 computes K=(SS/g^{x2·x4·s})^{x2}=g^{(x1+x3)·x2·x4·s}, and SS 8 computes K=(A/gx^{2x·x4·s})^{x4}=g^{(x1+x3)·x2·x4·s}. With the same keying material K, a session key (e.g., K-auth) can be derived K H(K), where H is a hash function. Therefore, by engaging in the user-centric authentication protocol, which may include one of a variety of the PAKE algorithms, each of display device 150 and SS 8 is able to derive K-auth (e.g., a session key). Details of the rest of the PAKE algorithms are not described herein for brevity.

In certain embodiments, one or more techniques may be used to configure SS 8 and/or display device 150 to engage in the user-centric authentication protocols described herein, for example, in response to an occurrence of a certain event or an action. For example, SS 8 may be configured to engage in a user-centric authentication protocol when SS 8 receives a certain trigger, such as when SS 8 is physically tapped more than a certain number of times. In one example, SS 8 may be configured to engage in a user-centric authentication protocol when SS 8 is tapped three times, in which case SS 8 transmits a request to display device 150 to engage in the user-centric authentication protocol. In certain embodiments, it may be advantageous to configure SS 8 to engage in the user-centric authentication protocol when it is tapped more than once, or receives certain predetermined actions, in order to avoid accidentally causing SS 8 to engage in the protocol (e.g., accidental tapping, such as when the user is playing a game and a ball hits SS 8). In certain embodiments, the SS 8 may be configured with haptics-related hardware and software for performing the techniques described above, as known to one of ordinary skill in the art.

In certain embodiments, the occurrence of a certain event may include SS 8 and/or display device 150 being in a certain pre-designated location. For example, SS 8 may be configured to engage in a user-centric authentication protocol when SS 8 and/or display device 150 are in a certain location. In certain embodiments, the location can be configurable by the user. In certain embodiments, if SS 8 and/or display device 150 are in the pre-designated location, the two devices may be able to share a secret in plain text in order to authenticate each other without having to engage in a user-centric protocol to derive K-auth. In certain embodiments, a user may indicate the selection of the preferred geographical location of display 150 and SS 8 via an app of the display device 150. In other embodiments, the preferred location may be determined (e.g., by a server) and provided to the display.

In certain embodiments, SS 8 and display device 150 (which may both be configured with Bluetooth and/or NFC related hardware and software for communication) may be configured to engage or initiate in the user-centric authentication protocol and/or the device-centric authentication protocol when the two devices are in close proximity to each other (e.g., when a signal strength threshold proximity requirement between the two is met).

Note that, as described above, in certain embodiments, display device 150 and SS 8 may first engage in the device-centric authentication protocol and, after a successful completion of that protocol, then engage in the user-centric authentication protocol. In certain other embodiments, display device 150 and SS 8 may first engage in the user-centric authentication protocol and, after a successful completion of that protocol, then engage in the device-centric authentication protocol. In certain cases engaging in the user-centric authentication protocol may be computationally more intensive. In such cases, engaging in the device-centric authentication protocol first may be more resource efficient for each device, in cases where the authentication ultimately fails.

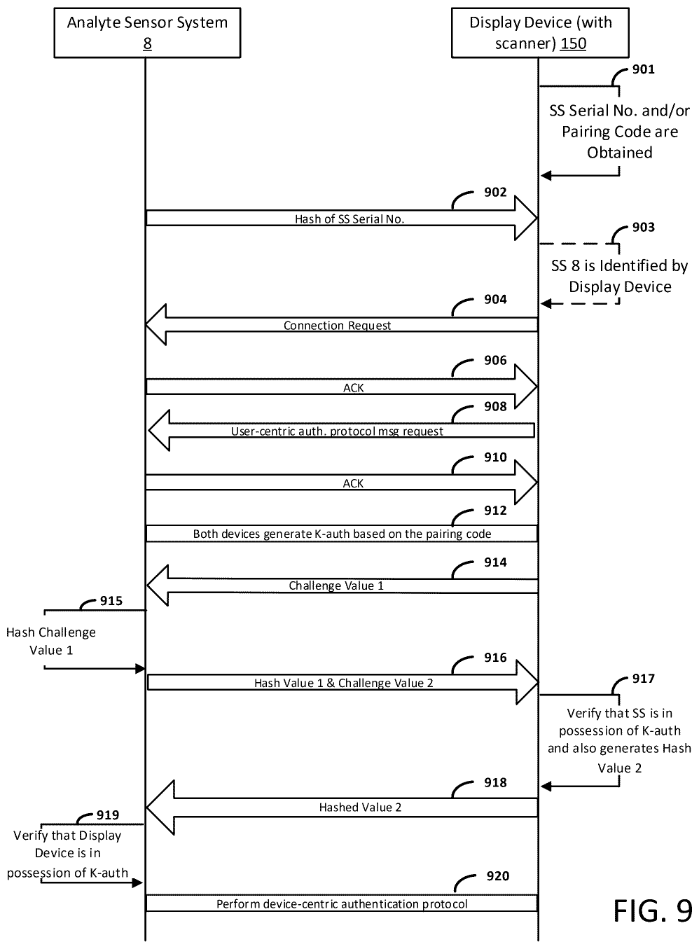

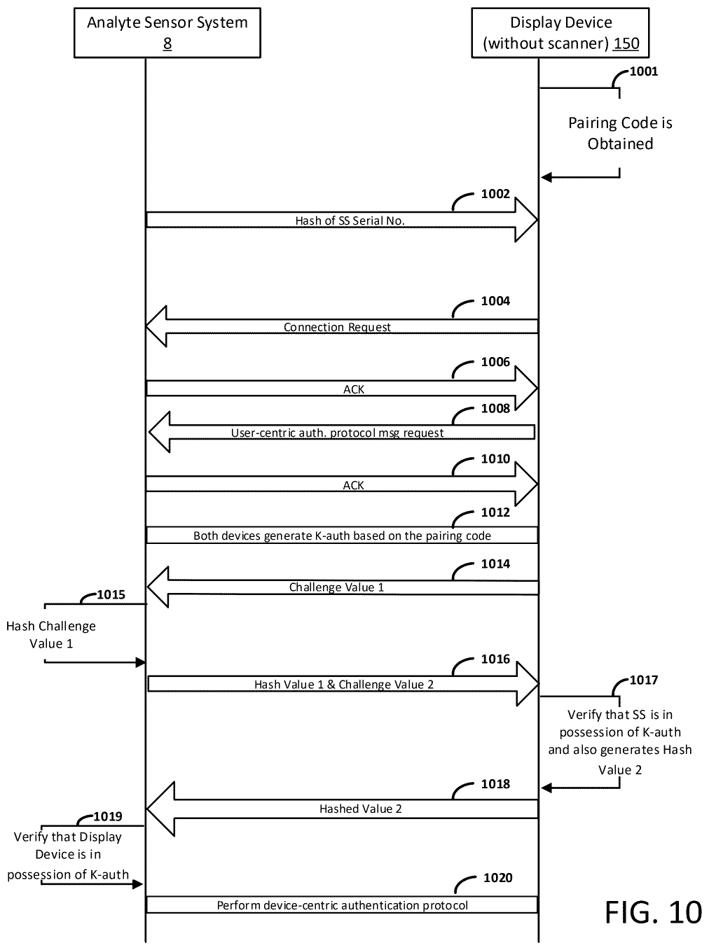

Also, note that in certain embodiments, instead of configuring SS 8 and display device 150 to perform a user-centric authentication protocol to derive K-auth, a QR code located on SS 8 or a packaging thereof may directly act as K-auth. In such embodiments, display device 150 (e.g., mobile device 120) is configured with the appropriate hardware and software to scan the QR code. After the user uses display device 150 to scan the QR code, display device 150 and SS 8 engage in a key verification protocol (e.g., FIGS. 6A-6C, steps 914-919 of FIG. 9, steps 1014-1019 of FIG. 10, etc.) to ensure that both sides are in possession of K-auth. Once each device verifies that the other is in possession of K-auth, they are authenticated.

### Combination of User-Centric and Device-Centric Authentication Protocols

In certain embodiments, the user-centric and device-centric authentication protocols may be combined, resulting in a single protocol. In such embodiments, instead of separately performing the user-centric and device-centric mutual authentication protocols, display device 150 and SS 8 (or any other partner devices as described above) are able to verify whether each party is trusted by server system 134 and whether the user trusts each device, by engaging in the single protocol. The user-centric and device-centric mutual authentication protocols may be combined in a number of ways.

In a first set of embodiments, the user-centric and device-centric mutual authentication protocols are combined by configuring a first device to send its certificate to the other as part of the data exchange that takes place when the first device and the second device engage in a PAKE algorithm. For example, display device 150 may transmit its certificate instead of a random exponent (i.e., instead of X₁ or X₂) that display device 150 would typically transmit to SS 8 initially (e.g., in what is referred to as “Round 1” of the J-PAKE algorithm, as described on page 10 of Hao). Similarly, display device 150 may transmit its certificate instead of a random exponent (e.g., instead of X₃ or X₄) that SS 8 would typically transmit to display device 150 in the initial round as described above (e.g., Round 1). Accordingly, in certain such embodiments, while engaging in, for example, the J-PAKE algorithm (which helps each of the two devices determine whether the other is trusted by the user), the two devices may also transmit their signed certificates to each other, thereby, allowing each of the two devices to also determine whether the other is trusted by a RCA (e.g., by verifying the signing authority).

In a second set of embodiments, the user-centric and device-centric authentication protocols are combined by configuring a first device to sign, using its public key, at least a portion of the information that is transmitted to a second device during Round 1 of the J-PAKE algorithm, as described on page 10 of Hao. In the second set of embodiments, with the same transmission, the first device is also configured to send its certificate to the second device. For example, display device 150 signs one or both of g^{x1} and g^{x2} and sends them together with the zero-knowledge proofs for X₁ or X₂ as well as display device 150's certificate to SS 8. Similarly, SS 8 signs one or both of g^{x3} and g^{x4} and sends them with the zero-knowledge proofs for X₃ or X₄ as well as SS 8's certificate to display device 150. Accordingly, in the second set of embodiments, each of the two devices is able to perform integrity verification, verify whether the other is trusted by a RCA, and also verify whether the user trusts the other device.

Performing a combination of the user-centric and device-centric authentication protocols, according to the first and second set of embodiments, may lead to higher resource and time efficiency. After engaging in the user-centric authentication protocol, each of display device 150 and SS 8 is further configured to engage in a key verification protocol to verify whether the other was also able to derive the same K-auth. FIGS. 6A-6C illustrate three alternative key verification protocols. If, after engaging in a key verification protocol, display device 150 and SS 8 are each able to verify that the other is in possession of the same K-auth, then each device concludes that it is able to trust the other device because the user also trusts the other device.

### Key Verification Protocols

FIG. 6A illustrates an example key verification protocol 600A, according to some embodiments. Note that some of the steps of key verification protocol 600A may be performed in parallel or overlap in time. Accordingly, the reference numbers assigned to the different steps of key verification protocol 600A may not be indicative of the order in which they are performed. In certain embodiments, example key verification protocol 600, is associated with user centric authentication and/or with combination of user and device centric authentication. In some embodiments, the key verification process is performed after the key generation based on the shared secret exchange.

At step 602, display device 150 transmits a first challenge value to SS 8. The first challenge value, in certain embodiments, is a randomly selected number.

At step 604, SS 8 hashes the first challenge value to generate a first hash value using a hashing algorithm as well as a shared key. In the example of FIG. 6A, the shared key is K-auth, which is the key derived during the user-centric authentication protocol (e.g., based on a pairing code).

At step 606, display device 150 hashes the first challenge value to generate a second hash value using the hashing algorithm and the shared key. The shared key used by display device 150 is K-auth. In alternative embodiments, the shared key may be a key from a partner device which may be shared with SS 8 and display device 150.

At step 608, SS 8 transmits the first hash value and a second challenge value to display device 150. In certain embodiments, the second challenge value is a randomly selected number.

At step 610, display device 150 verifies whether the first hash value and the second hash value are the same. If not, display device 150 concludes that SS 8 is not in possession of K-auth and fails out of key verification protocol 600A. If yes, display device 150 is able to conclude that SS 8 is in possession of K-auth and, therefore, can be trusted. If key verification protocol 600A fails, display device 150 provides a notification to the user that SS 8 was not authenticated. In certain embodiments, in the event of a failure, display device 150 ask the user to retry the authentication process.

At step 612, SS 8 hashes the second challenge value to generate a third hash value using the hash algorithm and the shared key.

At step 614, display device 150 transmits a fourth hash value to SS 8. For example, display device 150 hashes the second challenge value received from SS 8 to generate the fourth hash value using the hashing algorithm and K-auth.

At step 616, SS 8 verifies whether the third hash value and the fourth hash value are the same. If not, SS 8 fails out of key verification protocol 600A. If yes, SS 8 is able to conclude that display device 150 is in possession of K-auth and, therefore, can be trusted. In certain embodiments, because SS 8 does not have a display (and therefore cannot show a fail notification), the failure needs to be identified by display device 150.

FIG. 6B illustrates an example key verification protocol 600B, according to some embodiments. Note that some of the steps of key verification protocol 600B may be performed in parallel or overlap in time. Accordingly, the reference numbers assigned to the different steps of key verification protocol 600B may not be indicative of the order in which they are performed. Also note that, in certain embodiments, key verification protocol 600B may utilize SPEKE protocol.

At step 620, display device 150 computes a K′ by hashing K-auth and a random number R where, K′=H(K, R). In this formula, H( ) is a cryptographic hashing algorithm or function, which both display device 150 and SS 8 are configured with. H( ) takes as input K-auth and R and outputs K′. K′ may be referred to as an obfuscation key because it is generated for the purpose of obfuscating K-auth.

At step 622, display device 150 computes a first hash value, where the first hash value is H(K′), and further computes a second hash value, where the second hash value is H(H(K′)). In certain other embodiments instead of rehashing the first hash value to generate the second hash value, display device 150 may encrypt the first hash value resulting in a second hash value of E(H(K′)). E refers to encryption, such that E(H(K′)) refers to an encrypted version of (H(K′)).

At step 624, SS 8 computes K′ by hashing K-auth and R where, K′=H(K, R). In this formula, H( ) is the same hashing algorithm used by display device 150 at step 620. R is also the same R that is used by display device 150 as step 620.

At step 626, SS 8 computes a third hash value, where the third hash value is H(K′), and further computes a fourth hash value, where the fourth hash value is H(H(K′)). In certain other embodiments instead of rehashing the third hash value to generate the fourth hash value, SS 8 may encrypt the third hash value resulting in a fourth hash value of E(H(K′)).

At step 628, display device 150 transmits the second hash value to SS 8.

At step 630, SS 8 verifies whether the second hash value is the same as the fourth hash value. If yes, SS 8 is able to conclude that display device 150 is in possession of K-auth. If not, SS 8 concludes that display device 150 is not in possession of K-auth and fails out of key verification protocol 600B. If key verification protocol 600B fails, display device 150 provides a notification to the user that SS 8 was not authenticated. In certain embodiments, in the event of a failure, display device 150 ask the user to retry the authentication process.

At step 632, SS 8 transmits the third hash value, H(K′), to display device 150. In certain other embodiments instead of transmitting the third hash value, SS 8 may encrypt K′ to generate E(K′) and transmit an encrypted version of K′ to display device 150.

At step 634, display device 150 verifies whether the first hash value is the same as the third hash value. If yes, display device 150 is able to conclude that display device 150 is in possession of K-auth. If not, display device 150 concludes that SS 8 is not in possession of K-auth and fails out of key verification protocol 600B. In certain embodiments where, at step 632, SS 8 transmits E(K′) to display device 150, then at step 634, display device 150 is also configured to compute E(K′) to compare what is transmitted by SS 8, at step 632, with what is computed by display device 150. If key verification protocol 600A fails, display device 150 provides a notification to the user that SS 8 was not authenticated. In certain embodiments, in the event of a failure, display device 150 ask the user to retry the authentication process.

FIG. 6C illustrates an example key verification protocol 600C, according to some embodiments. Key verification protocol 600C may be used in conjunction with the embodiments of the device-centric authentication protocol described in relation to FIG. 4D. As described above, in contrast to FIG. 4C, in FIG. 4D, display device 150 and SS 8 are not configured to transmit signed messages to each other for integrity verification purposes. As such, in embodiments where display device 150 and SS 8 use the device-centric authentication protocol of FIG. 4D, the devices may use key verification protocol 600C, which allows each device to perform integrity verification during the key verification stage. Note that some of the steps of key verification protocol 600C may be performed in parallel or overlap in time. Accordingly, the reference numbers assigned to the different steps of key verification protocol 600C may not be indicative of the order in which they are performed.

Steps 640-646 are similar to steps 620-626 of FIG. 6B, respectively.

At step 648, display device 150 transmits the second hash value, H(H(K′)), as well as a signed version of the second hash value to SS 8. A signed version of the second hash value refers to the second hash value being encrypted with display device 150's DD-Priv.

At step 650, SS 8 verifies whether the second hash value is the same as the fourth hash value. If yes, SS 8 is able to conclude that display device 150 is in possession of K-auth. If not, SS 8 concludes that display device 150 is not in possession of K-auth and fails out of key verification protocol 600C. If key verification protocol 600C fails, display device 150 provides a notification to the user that SS 8 was not authenticated. In certain embodiments, in the event of a failure, display device 150 ask the user to retry the authentication process. SS 8 also verifies the integrity of the transmitting entity of the second hash value. For example, SS 8 uses display device 150's DD-pub from display device 150's verified certificate (e.g., verified at step 404 of FIG. 4D) and decrypts the signed second hash value. If what results from decrypting the signed second hash value is equal to the second hash value transmitted at step 648, then SS 8 is able to conclude that the transmitting entity of the second hash value is in fact display device 150 and not an attacker (e.g., after which SS 8 then sends an acknowledgement message to display device 150).

At step 652, SS 8 transmits the third hash value, H(K′), as well as a signed version of the third hash value to display device 150. A signed version of the third hash value refers to the third hash value being encrypted with SS 8's SS-Priv. Similar to FIG. 6B, in certain other embodiments, instead of transmitting the third hash value, SS 8 may encrypt K′ to generate E(K′) and transmit an encrypted version of K′ to display device 150.

At step 654, display device 150 verifies whether the first hash value is the same as the third hash value. If yes, display device 150 is able to conclude that SS 8 is in possession of K-auth. If not, display device 150 concludes that SS 8 is not in possession of K-auth and fails out of key verification protocol 600C. If key verification protocol 600C fails, display device 150 provides a notification to the user that SS 8 was not authenticated. In certain embodiments, in the event of a failure, display device 150 ask the user to retry the authentication process.

Display device 150 also verifies the integrity of the transmitting entity of the third hash value. For example, display device 150 uses SS 8's SS-Pub from SS 8's verified certificate (e.g., verified at step 408 of FIG. 4D) and decrypts the signed third hash value. If what results from decrypting the signed first hash value is equal to the third hash value transmitted at step 652, then display device 150 is able to conclude that the transmitting entity of the third hash value is in fact SS 8 and not an attacker. Similar to FIG. 6B, in embodiments where, at step 652, SS 8 transmits E(K′) to display device 150, then at step 654, display device 150 is also configured to compute E(K′) to compare what is transmitted at step 652 by SS 8 with what is computed by display device 150.

After engaging in the user-centric, the device-centric authentication, and/or the key verification protocols, or a combination thereof, each of display device 150 and SS 8 is able to determine that the other is trusted by a RCA, e.g., server system 134, as well as the user. Subsequently, SS 8 and display device 150 may establish a communication link for the exchange of data. In embodiments where SS 8 and display device 150 are configured with BLE protocols, the two devices engage in pairing and bonding as further described in relation to FIG. 7.

Performing both the user-centric and the device-centric mutual authentication protocols is advantageous because if only the user-centric mutual authentication protocol is used for authentication, an attacker may repeatedly attempt to engage in the user-centric mutual authentication protocol with one of SS 8 or display device 150 until the attacker is able to guess the correct K-auth through brute force (also referred to as a brute force attack). In such situations, by configuring each of SS 8 and display device 150 to further engage in the device-centric mutual authentication protocol, an attacker will ultimately fail to complete the authentication process because the attacker is not trusted by server system 134. In addition, in certain embodiments, in order to reduce the success rate of brute force attacks, the two devices may be configured with a throttling mechanism, as further described below.

On the other hand, in some examples, the display device 150 and SS 8 may be configured to perform the device-centric mutual authentication protocol. In such examples, if an attacker who has improperly obtained, for example, display device 150's certificate and private key, may able to authenticate with and gain access to SS 8. In such situations, by configuring each of SS 8 and display device 150 to further engage in the user-centric mutual authentication protocol, an attacker will ultimately fail to complete the authentication process because the attacker is not in possession of the shared secret (e.g., pairing code) that is used as part of the use-centric mutual authentication protocol.

Note that although it may be advantageous to perform both the user-centric and the device-centric authentication protocols, in certain embodiments, one of the protocols is performed for authentication purposes.

### Throttling

In order to reduce the success rate of brute force attacks described above with respect to an attacker's repeated attempts to engage in the user-centric authentication protocol, each of SS 8 and display device 150 may be configured with a throttling mechanism to manage or restrict the number of times the device can engage in the user-centric authentication protocol with a potentially malicious device. For example, a first device (e.g., an attacker's device) may keep trying to authenticate with a second device (e.g., display device 150 or SS 8). However, using a throttling mechanism, in certain embodiments, the second device may throttle the number of times the first device can initiate authentication with a wrong K-auth, or how many times the second device may acknowledge or participate in a request for authentication. For example, the second device may throttle the number of attempts by the first device to a certain defined number (e.g., one (1), two (2), etc.) and/or in a certain period of time. In certain embodiments, once the first device provides the wrong K-auth once, then the second device can refrain from engaging in any form of authentication with the first device for a certain period of time (e.g., 5 minutes). If, for example, after 5 minutes, the first device provides the wrong K-auth twice, then the first device may refrain from engaging in any form of authentication with the first device for a longer period of time (e.g., 10 minutes). In this example, the second device reduces the number of times the first device can initiate authentication with the wrong K-auth to 2 times in 15 minutes. In certain embodiments, a similar throttling mechanism may be used with respect to the device-centric authentication protocol, when, for example, a first device (e.g., an attacker) provides a certificate or a signature that cannot be authenticated by the second device. In some examples, the second device (e.g., the display device 150 or the SS 8) may provide an indication to the user about failed attempts to authenticate. For example, the DD 150 may provide a notification on the display of the DD 150 that an authentication could not be performed and further notify the user that another to authenticate the device(s) will take place in a predetermined amount of time. The above-mentioned notification related to failed attempt may be provided when SS 8, display device 150 and/or any other trusted partner devices initiates an authentication process and an attacker tries to attack the authentication process. It is contemplated that the user may take necessary initiatives (e.g., shutdown the device, notify legal authorities) to curtail such rogue attacks.

Note that, in certain embodiments, there is a wireless address (e.g., a BLE MAC (media access card) address or another identifier), associated with every device. In certain embodiments, when two devices engage in an authentication protocol, such as the user-centric or device-centric authentication protocols, data transmitted by each device comprises the wireless identifier. As such, for example, when a first device fails to authenticate with the second device once, the second device caches or stores the wireless address of the first device so that the second device can throttle any further attempts by the first device to authenticate during a certain period of time (e.g., 5 minutes), as described above.

### Single Device Rule

In certain cases, an attacker may use a malicious display device to engage in mutual authentication (e.g., user-centric and/or device-centric protocols) and/or key verification protocols with SS 8 while, for example, display device 150 (i.e., user's display device) is already engaged in one of such protocols with SS 8. In order to prevent the attacker from disrupting the authentication and/or key verification processes between SS 8 and display device 150, SS 8 may be configured to only engage in such protocols with a single display device at a time. As such, in certain embodiments, SS 8 may be configured to cache the wireless address, e.g., BLE MAC address, of display device 150 after the identification phase. Subsequently, if data packets with a different wireless address are received from a different display device, SS 8 is able to conclude that a device other than display device 150 is attempting to connect with SS 8, in which case SS 8 may drop such packets based on the single device rule.

### Further Verification of Identity of the SS by the Server

In certain embodiments, after and/or during display device 150 and SS 8 mutually authenticate each other, display device 150 further verifies the identity of SS 8 by sharing SS 8's SS-Pub or certificate (which includes SS-Pub), with server system 134. In certain embodiments, server system 134 stores public keys associated with SSs on the market, including SS 8. Therefore, if server system 134 receives SS 8's public key or certificate from more than one display device or from an untrusted display device or an untrusted partner device, or for multiple SSs (at least some of whose public keys may or may not be stored in the server) from the same display device (within a short period of time), server system 134 may be configured to determine that SS 8's public key has been compromised. For example, upon receiving SS 8's public key from display device 150, server system 134 may determine that another display device had also previously transmitted SS 8's public key to server system 134. In these situations, server system 134 may be configured to take one or more security measures, such as revoking SS 8's compromised certificate and issuing a new certificate. In such a scenario, the display device 150 may need to initiate a new authentication process with the SS 8 and the server system 134.

### Pairing and Bonding

Once display device 150 and SS 8 mutually authenticate each other using one or more authentication protocols, such as the user-centric and/or device-centric authentication protocols described above, the devices engage in pairing and/or bonding. In certain embodiments, SS 8 and display device 150 conform to various wireless protocols and standards (e.g., Bluetooth®, Bluetooth Low Energy (BLE), ANT protocols, Wi-Fi, Near Field Communication (NFC), etc.). In one example, the pairing and/or bonding performed between SS 8 and display device 150 are in accordance with the BLE standards. In some examples, according to the wireless protocols and standards (e.g., BLE secure mode pairing and bonding standards), pairing is the exchange of security features, such as Input/Output (IO) capabilities, requirements for Man-In-The-Middle (MITM) protection, etc. During pairing between two communication devices, the two devices may agree on a temporary key (TK), whose value may depend on the pairing method that is used. The two devices engage in securing the subsequent communication links for analyte data communication by generating various communication keys (e.g., short term key (STK), long term key (LTKs) to encrypt the communication links. In some examples, the devices may store additional information about each other during a bonding process, for example, after pairing. For example, after the exchange of security features and the encryption of the connection, the devices bond by exchanging the LTK and storing the LTK for later use. In other words, bonding refers to the creation of permanent security between devices. Further details regarding pairing and bonding can be found in the various specifications of such technologies (e.g., Bluetooth specification), which is incorporated herein by reference in its entirety. The specifications may be provided by the governing bodies of such technologies

FIG. 7 is a sequence diagram illustrating SS 8 and display device 150 engaging in pairing and bonding in accordance with the BLE standards. At step 702, SS 8 and display device 150 engage in pairing. In certain embodiments, during the pairing procedure (e.g., step 702), the two devices may engage in authenticated pairing for the purpose of providing protection against man-in-the-middle attacks. Typically, a display device 150 that is configured to perform authenticated pairing may prompt a user to manually enter a key (e.g., 6 digit passkey) to engage in pairing with SS 8. However, in certain embodiments, display device 150 may be configured to pair with SS 8 using application level security (e.g., pairing) protocols instead of using BLE protocols. For example, an application level pairing protocol may include a pairing protocol that is stored by a device as a result of instructions provided by an application executing on the device. In certain embodiments, when an application level pairing protocol is used, display device 150 may use K-auth or a version thereof, which was generated as a result of the two devices engaging in the user-centric authentication protocols described above, instead of requiring the user to manually input the 6 digit passkey.

At step 704, SS 8 and display device 150 engage in bonding. After pairing and bonding, SS 8 and display device 150 are ready to exchange data over a secure connection. For example, SS 8 may use the LTK to encrypt data, including analyte measurements associated with the user, for transmission to display device 150. Display device 150 may similarly encrypt data for transmission to SS 8. In other words, using the LTK, SS 8 and display device 150 are able to establish a secure connection for data exchange.

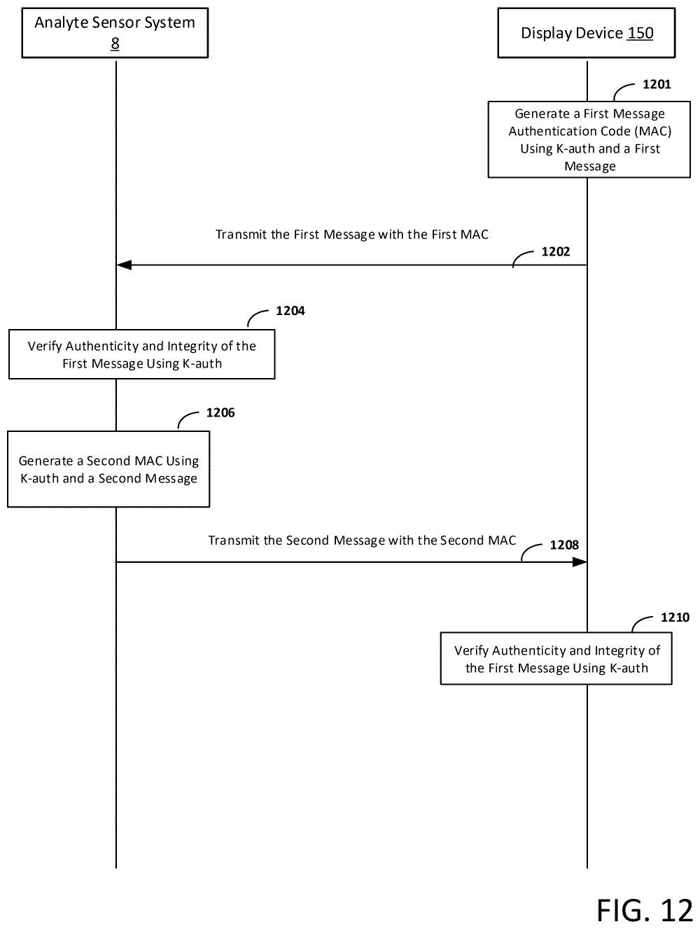

As described above, in certain embodiments, display device 150 may be configured to pair and/or bond with SS 8 using application level pairing and/or bonding protocols instead of or in addition to using BLE protocols. In certain such embodiments, display device 150 and SS 8 may be configured to use K-auth as an encryption key or use K-auth to derive an encryption key to be used for encryption instead of or in addition to using the BLE protocols to establish a LTK. As described in relation to FIG. 12, K-auth may not only be used for encryption (e.g., confidentiality) but also data integrity and authenticity when SS 8 and display device 150 exchange data (e.g., glucose data, sensor data, analyte data, commands, response, etc.). In certain embodiments, when K-auth is used by the two devices for data encryption, the key verification steps shown in FIGS. 6A-6C, steps 914-919 of FIG. 9, and steps 1014-1019 of FIG. 10 may not be performed. As such, the two devices can directly engage in encrypted (using K-auth) data exchange. The key verification steps may be skipped because if, for example, display device 150 is not in possession of K-auth, display device 150 will not be able to decrypt/encrypt communication with K-auth and, therefore, SS 8 is able to determine that display device 150 is not in possession of K-auth without having to perform the key verification protocols with display device 150. Skipping the key verification steps may lead to resource (compute and battery) efficiency, among other things. In certain embodiments, the key verification steps take place even though K-auth is used for encryption.

In certain embodiments, a first display device 150 (e.g., mobile device 120) may be configured to share K-auth (which may be used by the first display device 150 and SS 8 for encryption, data authenticity, and/or data integrity) with a second display device 150 (e.g., a smart watch, such as smart watch 140, or any other type of display device described herein). For example, the first and the second display devices 150 may be configured to connect to each other using certain communication protocols. In such embodiments, all application level security commands (e.g., including the agreed upon K-auth) associated with the first display device 150's communication with SS 8 may be communicated directly (or indirectly via a server) to the second display device 150 using suitable communication protocols. In such embodiments, the second display device 150 is able to use the same security commands to communicate with SS 8 without the need for the second display device 150 and SS 8 to re-establish the same security commands and agreements among each other. For example, the second display device 150 may use K-auth or a portion thereof for confidentiality (encrypting data using K-auth) and data integrity/authenticity (e.g., using MACs), as described in more detail with respect to FIG. 12. In another, example, the second display device 150 may directly engage in encrypted data communication with SS 8 instead of or in addition of key verification.

### Pass-Through Communications Between the Server System and SS

In certain embodiments, SS 8 and server system 134 may be configured to communicate through a pass-through communication path 183. As described above, pass-through communication path 183 refers to a communication path that is established between server system 134 and SS 8 through display device 150. In certain embodiments, communication over communication path 183 is encrypted such that display device 150 is not able to read or gain access to the data that is exchanged over communication path 183. In certain embodiments, communication over communication path 183 may be continuous and, in certain other embodiments, communication over communication path 183 may be periodic. In certain embodiments, communication over communication path 183 is encrypted with a key. In certain embodiments, the key is an AES key. In certain embodiments, both SS 8 and server system 134 are configured with or store this key. In certain embodiments, both SS 8 and server system 134 may store a plurality of keys as well as a plurality of corresponding index numbers, where each index number corresponds to a different key. In such embodiments, SS 8 may select a key from the plurality of keys, encrypt data for transmission to server system 134, and then transmit the encrypted data along with an unencrypted index number that identifies the key. In such embodiments, upon receiving the encrypted data and the unencrypted index number, server system 134 identifies the key associated with the unencrypted index number and then uses the key to decrypt the data.

In certain embodiments, instead of or in addition to storing one or more keys, SS 8 and server system 134 may engage in a certain key exchange algorithm to derive a key for encryption of data exchanged between SS 8 and server system 134. In certain embodiments, SS 8 and server system 134 engage in the DH key exchange algorithm. In certain embodiments, SS 8 and server system 134 may each be configured to perform a part of (e.g., half of) the DH key exchange algorithm. For example, SS 8 may store secret integers “t” and “b”. SS 8 may send to server system 134 “g^{t}”, where g is a primitive root modulo p. With “g^{t}”, SS 8 also sends an index for how “b”, which is also stored in server system 134, can be found by server system 134. Upon receiving “g^{t}” and the index number, server system 134 finds “b” based on the index number and calculates “g^{tb}”. At this point, both SS 8 and server system 134 are in possession of “g^{tb}”, which can be subsequently used for encryption of data exchanged between the two devices. In certain embodiments, instead of SS 8 and server system 134 engaging in the DH key exchange algorithm, they may engage in an elliptic curve DH (“ECDH”) key exchange algorithm. In certain embodiments, SS 8 and server system 134 may each be configured to perform a part of (e.g., half of) the ECDH key exchange algorithm. Yet in another embodiment, SS 8 and server system 134 may each perform full ECDH key exchange algorithm.

### Security Measures for Reconnections

In certain embodiments, for power saving purposes, SS 8 may periodically exchange data with display device 150 (e.g., by switching between a sleep mode and an operational mode periodically). For example, in certain embodiments, SS 8 may “wake up” every few minutes (e.g., five minutes) to exchange data with display device 150 but go into sleep mode in-between the five minute intervals. In order to reduce the likelihood of an attacker attempting to communicate with, for example, SS 8 by replaying certain data transmissions previously sent to SS 8 by display device 150, the two device may be configured to perform one or more security measures during these periodic reconnections.

FIG. 8 is a sequence diagram illustrating a number of security measures SS 8 and display device 150 may, in certain embodiments, take during periodic reconnections.

At step 802, SS 8 and display device 150 engage in a key verification protocol at the periodic reconnections (e.g., at the start of the periodic reconnections). For example, in certain embodiments, every five minutes, SS 8 and display device 150 engage in the same key verification protocol the two devices previously engaged in or a different key verification protocol. As an example, SS 8 and display device 150 may be configured to engage in key verification protocol 600A during their first connection (i.e., the first time they perform key verification) and then, at subsequent reconnections, engage in key verification protocol 600B. This may deter the attacker, who may have tried to attack (and possibly succeeded) at the first connection, but would now fail in the second connection, because of the use of a different key verification protocol by the SS 8 and display device 150.

At step 804, SS 8 and display device 150 may optionally rekey K-auth (i.e., derive a new K-auth), for example, at periodic interval connections. K-auth may be rekeyed in one of a number of ways. In certain embodiments, over a secured connection (encrypted by LTK), SS 8 and display device 150 may rekey K-auth by generating a random key or using an application level K-auth from a plurality of keys that are reserved (e.g., stored in devices) for this purpose. An application level K-auth refers to a K-auth that is stored by a device as a result of instructions provided by an application executing on the device and not generated as a result of the device engaging in, for example, a user-centric authentication protocol (e.g., J-PAKE) with another device to derive the key. In certain embodiments, only one of SS 8 and display device 150 may store one or more K-auths, in which case the device that stores the one or more K-auths may select one of the one or more K-auths and transmit it over a secure connection to the other device. In certain other embodiments, both devices may be configured to store one or more K-auths. In such embodiments, the devices are configured to select the same K-auth at different reconnections, for example, by using a counter or some other mechanism. In certain other embodiments, instead of or in addition to using a random or application level K-auth for rekeying, SS 8 and display device 150 may engage in a user-centric authentication protocol to derive a new K-auth. Rekeying K-auth periodically may be advantageous in cases where an attacker is able to retrieve a current K-auth by engaging brute force attacks.

After rekeying K-auth, the new K-auth is used in a key verification protocol that the devices are configured to engage in during the next periodic reconnection.

At step 806, SS 8 and display device 150 engage in an exchange of information over a secure connection. For example, every few minutes, SS 8 and display device 150 engage in a key verification protocol, optionally rekey K-auth, and then exchange data (e.g., analyte measurements) that is encrypted using LTK among each other.

### Nonce

In certain embodiments, in order to reduce the likelihood of replay attacks, both SS 8 and display device 150 may be configured to include a nonce in each transmission (e.g., in one or more data packets) to the other device. A nonce, refers to a number that is used only once. For example, in certain embodiments, display device 150 and SS 8 may each be configured with a counter. With each transmission, the counter may be incremented by “1.” As such, in that example, each time a device transmits data to the other it increments its counter and uses the counter's value as a nonce in the next transmission. Thus, if a receiving device receives a nonce twice, it may be configured to determine that a replay attack has taken place.

### EXAMPLE EMBODIMENTS

FIGS. 9-12 illustrate example embodiments of how the protocols discussed above (or varied versions thereof) may be utilized by SS 8 and display device 150 and other entities in the system 100 to securely identify and authenticate each other, thereby, enabling the two devices to exchange data in a secure and confidential manner.

FIG. 9 illustrates the use of the user-centric mutual authentication protocol followed by the use of the device-centric protocol by SS 8 and a display device 150 that is configured with software and hardware (e.g., camera) for scanning a QR code (e.g., a bar code that may include a SIN of SS 8, pairing code, etc.). For example, in certain embodiments of FIG. 9, display device 150 may be a mobile device, such as mobile device 120, that has a camera for scanning information, such as QR codes.

At step 901, display device 150 obtains a pairing code and optionally a SIN associated with SS 8. For example, as introduced above, a SIN and a paring code may be located on the SS 8 or on a packaging of the SS 8 or on other devices associated with the SS 8. In certain embodiments, a user may use display device 150 to obtain the SIN and/or the paring code, such as by scanning a QR code. In certain other embodiments, the user may choose not to use the scanning capabilities of display device 150 and instead input information manually. In such embodiments, however, the user may be allowed or enabled to input the pairing code and not the SIN. As a result, in embodiments where the user does not use scan the QR code, the display device 105 may obtain the pairing code through its user interface. In some embodiments, a SIN may also be entered using the user interface of the display device 150.

At step 902, SS 8 advertises a hash of the SIN. For example, SS 8 may use a hashing algorithm to generate the hash of the SIN that SS 8 may periodically advertise (e.g., subsequent to being placed on the user's body and activated). SS 8 may advertise the hash of the SIN by including the hash of the SIN in data packets that comprise additional information, such as manufacturer related information.

At optional step 903, display device 150 identifies SS 8 (or confirms the identify of SS 8). For example, display device 150 hashes (using the same hashing algorithm SS 8 used to generate the hash of the SIN) the SIN that was obtained at step 901 to determine whether the hashed version of the scanned SIN is the same as the hash of the SIN that is received from SS 8 at step 902. If they match, then display device 150 is able to identify SS 8 as the correct SS from among potentially a number of other devices that may also be advertising their SINs or a version thereof. Note that in embodiments where the user chooses not to scan the SIN and instead manually inputs the pairing code, step 904 may not be performed.

At step 904, display device 150 transmits a connection request to SS 8.

At step 906, SS 8 acknowledges the connection request.

At step 908, display device 150 transmits a message request to SS 8 to engage in a user-centric authentication protocol. For example, the message request indicates the display device 150's desire to engage in, for example, a PAKE protocol (e.g., a type of user-centric mutual authentication protocol) with SS 8. There are various phases of the PAKE protocol (e.g., phase 0, 1, 2, etc), and in one example, the message may indicate a phase (e.g., phase 0).

At step 910, SS 8 acknowledges the request.

At step 912, display device 150 and SS 8 engage in the user-centric authentication protocol (e.g., PAKE), as previously discussed in relation to FIG. 5. Note that SS 8 and display device 150 engage in, e.g., the PAKE protocol to generate a K-auth by using the pairing code. The display device 150 has obtained the pairing code at step 901, as described above, and SS 8 is pre-configured with the pairing code during the manufacturing process. In some cases, the SIN may also be used in generating the K-auth.

Steps 914-919 are performed so that each of the two devices can ensure that the other is in possession of K-auth.

At step 914, display device 150 transmits a first challenge value (“Challenge Value 1”) to SS 8. Challenge Value 1 is, in some embodiments, a randomly generated number.

At step 915, SS 8 hashes Challenge Value 1 to generate a hash value (“Hash Value 1”). For example, SS 8 hashes Challenge Value 1 using a hashing algorithm and K-auth to generate Hash Value 1. The hashing algorithm is known to both SS 8 and display device 150. In some cases the k-auth may also be called an application (app) key.

At step 916, SS 8 transmits Hash Value 1 and a second challenge value (“Challenge Value 2”) to display device 150. Challenge Value 2 is, in some embodiments, a randomly generated number.

At step 917, display device 150 verifies whether SS 8 is in possession of K-auth and also generates Hash Value 2 to transmit to SS 8. More specifically, display device 150 uses the same hashing algorithm that is known to SS 8 as well as K-auth to hash Challenge Value 1 (which was already known to display device 150). If the resulting hash value is the same as Hash Value 1 received from SS 8 at step 916, then display device 150 has been able to verify that SS 8 is in possession of K-auth and that display device 150 is communicating with the correct device. If not, then display device 150 terminates the protocol. Once display device 150 has verified that SS 8 is in possession of K-auth, display device 150 uses the same hashing algorithm and K-auth to hash Challenge Value 2 (received from the SS 8), resulting in a hash value (“Hash Value 2”) to transmit to SS 8.

At step 918, display device 150 transmits Hash Value 2 to SS 8.

At step 919, SS 8 verifies whether display device 150 is in possession of K-auth. More specifically, SS 8 uses the same hashing algorithm as well as K-auth to hash Challenge Value 2 (which is already known by the SS 8). If the resulting hash value is the same as Hash Value 2 received from display device 150 at step 918, then SS 8 has been able to verify that display device 150 is in possession of K-auth and, therefore, SS 8 is communicating with the correct device. If not, then SS 8 terminates the protocol.

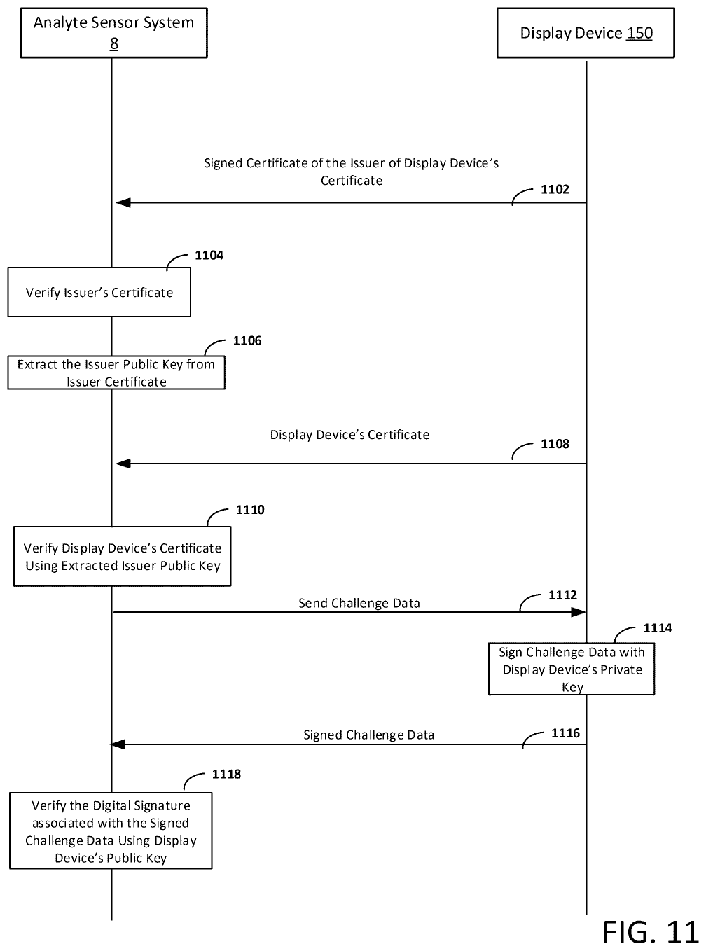

At step 920, once display device 150 and SS 8 have completed engaging in the user-centric mutual authentication protocol (e.g., the PAKE protocol) and verified that they each are in possession of K-auth, the two devices then engage in performing a device-centric authentication protocol, such as protocols that conform to the PKI scheme described earlier. A detailed example of such a device-centric authentication protocol is provided in FIG. 11 (as well as certain portion of FIGS. 4A-4C and the corresponding description).

FIG. 10 illustrates the use of the user-centric mutual authentication protocol followed by the use of the device-centric protocol by SS 8 and a display device 150 that is not configured with any hardware for scanning the SIN of SS 8. An example of such a display device 150 is display device 110 or any other type of display device that is unable to scan the SIN of SS 8.

At step 1001, display device 150 obtains the pairing code of SS 8. A user may manually input the pairing code of SS 8 into a user interface of display device 150.

At step 1002, SS 8 advertises a hash of the SIN. Step 1002 is similar to step 902 of FIG. 9. However, in the embodiments of FIG. 10, because display device 150 does not have access to SS 8's SIN, display device 150 may disregard the hash of the SIN received from SS 8 at step 1002. In such embodiments, display device 150 is not able to determine whether SS 8 is the correct device from among a number of devices advertising their SINs.

Steps 1004-1020 are similar to steps 904-920 of FIG. 9.

FIG. 11 illustrates a display device 150, a SS 8 and/or other entities in the system 100 engaging in a device-centric mutual authentication protocol.

At step 1102, display device 150 transmits a signed certificate of an issuer of display device 150's certificate (also referred to as a digital certificate or token). The issuer may be a SCA (subordinate certificate authority) having an intermediary certificate that has been signed by the RCA (root certificate authority, e.g., server system 134). The issuer's certificate includes the issuer's public key. Note that the issuer's certificate is signed with RCA-Priv (e.g., server system 134's private key, as described above). In certain embodiments, the issuer's certificate as well as display device 150's own certificate (including display device 150's public key (DD-Pub)) and private key (DD-Priv) are stored in display device 150 during the manufacturing process. In certain embodiments, display device 150 is able to receive the issuer's signed certificate RCA as well as display device 150's own certificate (including display device 150's public key) and private key from server system 134 (e.g., over network 190 in FIG. 1) in response to the user downloading analyte sensor application 121 and then signing up (e.g., by supplying the user's email address). In some embodiments, the various certificates and keys may be provided during the manufacturing process to the devices.

At step 1104, SS 8 authenticates or verifies the signed certificate of the issuer (e.g., a manufacturer of the system 100). For example, SS 8 uses RCA-PUB to decrypt a digital signature (in the issuer's signed certificate) that was generated using RCA-priv. Additional details regarding how an intermediary certificate can be authenticated are provided in relation to FIG. 4B and its respective description. Authenticating the issuer's certificate refers to confirming that the issuer's certificate was in fact signed by the RCA.

At step 1106, SS 8 extracts the issuer's public key from the issuer's certificate.

At step 1108, display device 150 transmits its own certificate, signed by the issuer (e.g. the manufacturer), to SS 8.

At step 1110, SS 8 verifies display device 150's certificate using the issuer's public key. Details regarding how display device 150's certificate can be verified by SS 8 are provided in relation to FIGS. 4A (e.g., step 404) and 4B and their respective description.

Steps 1112-1118 are performed by the two devices so that SS 8 can determine whether display device 150 holds a private key (DD-Priv) that matches the public key (DD-Pub) certificate of display device 150 that display device 150 transmitted to SS 8 at step 1108.

At step 1112, SS 8 transmits challenge data (e.g., random data) to display device 150 for the purpose of determining whether or not it was actually display device 150 itself that transmitted display device 150's certificate at step 1108 (i.e., whether display device 150 holds a private key (DD-Priv) that matches the public key (DD-Pub) certificate of display device 150 that display device 150 transmitted to SS 8 at step 1108).

At step 1114, display device 150 signs the challenge data with display device 105's private key (DD-Priv).

At step 1116, display device 150 transmits the signed challenged data to SS 8.

At step 1118, SS 8 verifies the digital signature associated with the signed challenge data using the display device 150's public key from device's public key certificate which was received earlier. As described above, verifying or authenticating display device's 150 digital signature allows SS 8 to determine whether or not it was actually display device 150 itself that transmitted display device 150's certificate at step 1108. In other words, SS 8 verifies the integrity of the transmitting entity of display device 150's certificate, which involves verifying that the transmitter is in possession of DD-Priv. Note that the transmitting entity of display device 150's certificate is the entity that transmitted the display device 150's certificate at step 1108. Because no other device than display device 150 can be in possession of DD-Priv, by ensuring that the transmitting entity is in possession of DD-priv, SS 8 may conclude that the transmitting entity of display device 150's credentials is in fact display device 150. Additional details regarding verifying display device 105's digital signature are provided with respect to FIG. 4C and its corresponding description.

Although not shown, in a symmetric fashion, SS 8 similarly transmits any relevant certificates (e.g., SS 8's certificate, a certificate of the issuer of SS 8's certificate) to display device 150 to allow display device 150 to verify whether SS 8 is trusted by the RCA. In addition, display device 150 transmits challenge data for SS 8 to sign using SS 8's private key. Upon receiving the signed challenge data, display device 150 is similarly able to verify the integrity of the transmitting entity of SS 8's certificate, which involves verifying that the transmitter is in possession of SS-Priv (i.e., verifying that it was actually SS 8 itself that transmitted SS 8's certificate). Note that the device-centric authentication (e.g., PKI certificate exchange process described in FIG. 11) may take place once per user-centric authentication (e.g., the PAKE process).

FIG. 12 illustrates an example use of K-auth for verifying data authenticity/integrity and providing confidentiality when SS 8 and display device 150 begin exchanging information (e.g., glucose values, sensor data, analyte data, etc.). For example, after display device 150 and SS 8 mutually authenticate each other using one or more authentication protocols, such as the user-centric and/or device-centric authentication protocols described above, the two devices may engage in pairing and/or bonding, as illustrated in FIG. 7. As part of the pairing and/or bonding (which may be performed using BLE protocols or application level protocols), the two devices may agree to utilize K-auth for encrypting data exchanged between the two devices and verifying the exchanged data's authenticity/integrity (through the use of message authentication codes (MACs)), as described below. As discussed, K-auth may be derived, in some embodiments, as a result of display device 150 and SS 8 engaging in a user-centric mutual authentication protocol (e.g., PAKE protocol), as shown in FIGS. 5, 9, and 10.

At step 1201, display device 150 uses a MAC algorithm that takes as input a first message as well as K-auth to generate a first MAC. In certain embodiments, the first message may include any data (e.g., data request command, request for sensor status, request for calibration data, request of algorithm status, etc.) that display device 150 may be configured to communicate with SS 8. In certain embodiments, display device 150 may also encrypt the first message using K-auth. In certain embodiments, display device 150 and SS 8 may agree to use a part (e.g., half) of K-auth for generating MACs and the other part (e.g., half) for encrypting the corresponding messages. For example, display device 150 may use half of K-auth for generating the first MAC based on the first message and use the other half for encrypting the first message.

At step 1202, display device 150 transmits the first message with the first MAC to SS 8.

At step 1204, SS 8 verifies the authenticity and integrity of the first message using K-auth. In embodiments where the first message is encrypted using K-auth or a portion thereof, SS 8 first decrypts the first message. Next, SS 8 uses the same MAC algorithm, which SS 8 may be configured with during the manufacturing process, to take the first message and K-auth as input and generate a MAC as output. If the generated MAC is the same as the first MAC received at step 1202, then SS 8 is able to verify that the first message, in fact, came from display device 150 (because only display device 150 should be in possession of K-auth) and that the message was not tampered with during transmission.

In embodiments where the first message is not encrypted using K-auth, SS 8 does not have to decrypt the first message before verifying its authenticity and integrity. In embodiments where a part of K-auth is used by display device 150 for encrypting the first message and the other part for generating the first MAC, SS 8 first decrypts the first message using the same part of K-auth that was used for the encryption and then verifies the authenticity and integrity of the first message using the other part of K-auth. In certain embodiments, both display device 150 and SS 8 are configured with the same algorithm that configures each device to be able to determine which portion of the K-auth to use for encryption and which portion to use for generating MACs.

At step 1206, SS 8 generates a second MAC using K-auth and a second message. SS 8 uses the same MAC algorithm described above to perform similar operations as display device 150 at step 1201. The second message may include any data (e.g., analyte data, sensor data or any data that is being requested by the display device 150 using MAC) that is typically sent by SS 8 to display device 150 as part of SS 8's operations.

At step 1208, SS 8 transmits the second message with the second MAC to display device 150.

At step 12010, display device 150 verifies the authenticity and the integrity of the second message by performing operations similar to the operations performed by SS 8 at step 1204.

The embodiments described herein provide technical solutions to technical problems associated with certain prior art security protocols used for purposes of identification, authentication, key verification, etc. For example, certain embodiments described herein relate to a number of identification protocols designed to allow a display device to effectively identify a SS and to reduce the likelihood of an attacker being able to obtain any information during the identification process that may become useful in impersonating the SS or display device. Further, certain embodiments described herein relate to a number of authentication and key verification protocols that the SS and display device may engage in, after the identification phase, to allow each device to verify whether the other device is trusted by a RCA and/or a user of the device.

Each of these non-limiting examples can stand on its own or can be combined in various permutations or combinations with one or more of the other examples. The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method of secure wireless communications between a sensor system and a display device, comprising:
performing, at the sensor system, a user-centric mutual authentication protocol with the display device to determine whether the display device is trusted by a user of the sensor system;
after determining that the display device is trusted by the user of the sensor system, performing, at the sensor system, a device-centric mutual authentication protocol with the display device to determine whether the display device is trusted by a diabetes trust management system, wherein performing the device-centric mutual authentication protocol comprises:
receiving one or more credentials from the display device to verify whether the display device is trusted by the diabetes trust management system, wherein the one or more credentials of the display device comprise a credential token of the display device;
verifying the one or more credentials of the display device, wherein the verifying comprises authenticating the credential token of the display device;
transmitting one or more credentials of the sensor system to the display device for verification of the one or more credentials of the sensor system by the display device; and
determining, at the sensor system, that the display device is trusted by the diabetes trust management system after transmitting of the one or more credentials of the sensor system; and
after performing the device-centric mutual authentication protocol is successful, transmitting, at the sensor system, analyte data to the display device.

2. The method of claim 1, wherein:
performing the user-centric mutual authentication protocol comprises performing a password authenticated key exchange (PAKE) algorithm with the display device using a shared key; and
performing the PAKE algorithm comprises deriving an authorization key.

3. The method of claim 2, further comprising:
prior to performing the device-centric mutual authentication protocol, performing, at the sensor system, a key verification protocol with the display device to verify whether the display device is also in possession of the authorization key, wherein performing the key verification protocol with the display device comprises:
receiving, at the sensor system, a first challenge value;
hashing, at the sensor system, the first challenge value to generate a first hash value using a hashing algorithm and the authorization key;
transmitting, to the display device, the first hash value and a second challenge value for the display device to verify whether the sensor system is in possession of the authorization key;
hashing, at the sensor system, the second challenge value to generate a third hash value using the hashing algorithm and the authorization key;
receiving, at the sensor system, a fourth hash value from the display device;
verifying, at the sensor system, whether the third hash value and the fourth hash value are the same; and
determining, at the sensor system, that the display device is trusted by the user of the sensor system and is in possession of the authorization key upon verifying that the third hash value and the fourth hash value are the same.

4. The method of claim 2, wherein transmitting the analyte data to the display device comprises encrypting the analyte data with the authorization key or a portion of the authorization key.

5. The method of claim 4, further comprising:
receiving, at the sensor system, a communication that is encrypted with the authorization key from a second display device without having engaged in the user-centric mutual authentication protocol with the second display device; and
the second display device is configured with the authorization key by the display device.

6. The method of claim 2, wherein transmitting the analyte data to the display device comprises:
generating a first message authentication code (MAC) using the analyte data, a MAC algorithm, and the authorization key or a portion of the portion of the authorization key; and
transmitting the analyte data with the first MAC to the display device, wherein the display device:
receives the analyte data and the first MAC;
uses the analyte data and the authorization key or a portion of the authorization key to generate a second MAC; and
verifies authenticity and integrity of the analyte data by determining that the first MAC and the second MAC are identical.

7. The method of claim 1, wherein:
the one or more credentials of the display device comprise an issuer credential token of an issuer of the credential token of the display device; and
verifying the one or more credentials of the display device comprises authenticating the issuer credential token.

8. The method of claim 1, further comprising:
transmitting a first challenge data to the display device;
receiving a signed version of the first challenge data including a digital signature generated by the display device using a private key associated with the display device; and
verifying authenticity of the digital signature by using a public key associated with the display device to decrypt the digital signature.

9. The method of claim 1, wherein performing the user-centric mutual authentication protocol is initiated only after the sensor system receives multiple haptic taps.

10. The method of claim 1, wherein performing the user-centric mutual authentication protocol is initiated only when at least one of the sensor system or the display device is in a designated location.

11. The method of claim 1, further comprising:
hashing a first serial identification number (SIN) of the sensor system using a SIN hashing algorithm;
transmitting a hash of the first SIN to the display device, wherein the display device:
uses the SIN hashing algorithm to hash a second SIN obtained by the display device through scanning a quick response (QR) code on the sensor system;
compares the hash of the first SIN received from the sensor system with a hash of the second SIN generated through hashing the second SIN obtained by the display device through scanning the QR code; and
confirms an identify of the sensor system upon determining that the hash of the first SIN and the hash of the second SIN are identical.

12. A sensor system for measuring blood glucose levels, comprising:
one or more processors configured to execute instructions stored on one or more memories and to cause the sensor system to:
perform a user-centric mutual authentication protocol with a display device to determine whether the display device is trusted by a user of the sensor system;
perform, after the determination that the display device is trusted by the user of the sensor system, a device-centric mutual authentication protocol with the display device to determine whether the display device is trusted by a diabetes trust management system, wherein, to perform the device-centric mutual authentication protocol, the one or more processors are configured to cause the sensor system to:
receive one or more credentials from the display device to verify whether the display device is trusted by the diabetes trust management system, wherein the one or more credentials of the display device comprise a credential token of the display device;
verify the one or more credentials of the display device, wherein, to verify the one or more credentials of the display device, the one or more processors are configured to cause the sensor system to authenticate the credential token of the display device;
transmit one or more credentials of the sensor system to the display device for verification of the one or more credentials of the sensor system by the display device; and
determine that the display device is trusted by the diabetes trust management system after transmission of the one or more credentials of the sensor system; and
transmit, after performance of the device-centric mutual authentication protocol is successful, analyte data to the display device.

13. The sensor system of claim 12, wherein:
to perform the user-centric mutual authentication protocol, the one or more processors are configured to cause the sensor system to perform a password authenticated key exchange (PAKE) algorithm with the display device using a shared key; and
to perform the PAKE algorithm, the one or more processors are configured to cause the sensor system to derive an authorization key.

14. The sensor system of claim 13, wherein the one or more processors are further configured to cause the sensor system to:
perform, prior to performance of the device-centric mutual authentication protocol, a key verification protocol with the display device to verify whether the display device is also in possession of the authorization key, wherein, to perform the key verification protocol with the display device, the one or more processors are configured to cause the sensor system to:
receive a first challenge value;
hash the first challenge value to generate a first hash value using a hashing algorithm and the authorization key;
transmit the first hash value and a second challenge value for the display device to verify whether the sensor system is in possession of the authorization key;
hash the second challenge value to generate a third hash value using the hashing algorithm and the authorization key;
receive a fourth hash value from the display device;
verify whether the third hash value and the fourth hash value are the same; and
determine that the display device is trusted by the user of the sensor system and is in possession of the authorization key upon verification that the third hash value and the fourth hash value are the same.

15. The sensor system of claim 13, wherein, to transmit the analyte data to the display device, the one or more processors are configured to cause the sensor system to encrypt the analyte data with the authorization key or a portion of the authorization key.

16. The sensor system of claim 15, wherein:
the one or more processors are further configured to cause the sensor system to receive a communication that is encrypted with the authorization key from a second display device without having engaged in the user-centric mutual authentication protocol with the second display device; and
the second display device is configured with the authorization key by the display device.

17. The sensor system of claim 13, wherein, to transmit the analyte data to the display device, the one or more processors are configured to cause the sensor system to:
generate a first message authentication code (MAC) using the analyte data, a MAC algorithm, and the authorization key or a portion of the portion of the authorization key; and
transmit the analyte data with the first MAC to the display device.

18. The sensor system of claim 12, wherein:
the one or more credentials of the display device comprise an issuer credential token of an issuer of the credential token of the display device; and
to verify the one or more credentials of the display device, the one or more processors are configured to cause the sensor system to authenticate the issuer credential token.

19. The sensor system of claim 12, wherein the one or more processors are further configured to cause the sensor system to:
transmit a first challenge data to the display device;
receive a signed version of the first challenge data including a digital signature generated by the display device using a private key associated with the display device; and
verify authenticity of the digital signature, wherein, to verify the authenticity of the digital signature, the one or more processors are configured to cause the sensor system to use a public key associated with the display device to decrypt the digital signature.

20. A method of secure wireless communications in a health management system comprising a sensor system and a display device, the method comprising:
performing a user-centric mutual authentication protocol with the display device to determine whether the display device is trusted by a user of the sensor system;
after determining that the display device is trusted by the user of the sensor system, performing a device-centric mutual authentication protocol with the display device to determine whether the display device is trusted by a diabetes trust management system, wherein performing the device-centric mutual authentication protocol comprises:
receiving one or more credentials from the display device to verify whether the display device is trusted by the diabetes trust management system, wherein the one or more credentials of the display device comprise a credential token of the display device;
verifying the one or more credentials of the display device, wherein the verifying comprises authenticating the credential token of the display device;
transmitting one or more credentials of the sensor system to the display device for verification of the one or more credentials of the sensor system by the display device; and
determining that the display device is trusted by the diabetes trust management system after transmitting of the one or more credentials of the sensor system; and
after performing the device-centric mutual authentication protocol is successful, transmitting analyte data to the display device.
